SEQUENCE LISTING (4 sequences)

SEQ ID NO: 1 - protein, 941 aa, Mus musculus
  MISC_FEATURE (1)..(941): Rubicon (mouse)
MRPEGAGMDLGGGDGERLLEKSRREHWQLLGNLKTTVEGLVSANCPNVWSKYGGLERLCRDMQNILYHGLIHDQVCCRQADYWQFVKDIRWLSPHSALHVEKFISLHESDQSDTDSVSERAVAELWLQHSLQCHCLSAQLRPLLGDRQYIRKFYTETAFLLSDAHVTAMLQCLEAVEQNNPRLLAQIDASMFARKQESPLLVTKSQSLTALPGSTYTPPASYAQHSYFGSSSSLQSMPQSSHSSERRSTSFSLSGPSWQPQEDRECLSPAETQTTPAPLPSDSTLAQDSPLTAQEMSDSTLTSPLEASWVSSQNDSPSDVSEGPEYLAIGNPAPHGRTASCESHSSNGESSSSHLFSSSSSQKLESAASSLGDQEEGRQSQAGSVLRRSSFSEGQTAPVASGTKKSHIRSHSDTNIASRGAAEGGQYLCSGEGMFRRPSEGQSLISYLSEQDFGSCADLEKENAHFSISESLIAAIELMKCNMMSQCLEEEEVEEEDSDREIQELKQKIRLRRQQIRTKNLLPAYRETENGSFRVTSSSSQFSSRDSTQLSESGSAEDADDLEIQDADIRRSAVSNGKSSFSQNLSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMVVPSRILRKWDFSKYYVSNFSKDLLLKIWNDPLFNVQDINSALYRKVKLLNQVRLLRVQLYHMKNMFKTCRLAKELLDSFDVVPGHLTEDLHLYSLSDLTATKKGELGPRLAELTRAGAAHVERCMLCQAKGFICEFCQNEEDVIFPFELHKCRTCEECKACYHKTCFKSGRCPRCERLQARRELLAKQSLESYLSDYEEEPTEALALEATVLETT

SEQ ID NO: 2 - protein, 972 aa, Homo sapiens
  MISC_FEATURE (1)..(972): Rubicon (human)
MRPEGAGMELGGGEERLPEESRREHWQLLGNLKTTVEGLVSTNSPNVWSKYGGLERLCRDMQSILYHGLIRDQACRRQTDYWQFVKDIRWLSPHSALHVEKFISVHENDQSSADGASERAVAELWLQHSLQYHCLSAQLRPLLGDRQYIRKFYTDAAFLLSDAHVTAMLQCLEAVEQNNPRLLAQIDASMFARKHESPLLVTKSQSLTALPSSTYTPPNSYAQHSYFGSFSSLHQSVPNNGSERRSTSFPLSGPPRKPQESRGHVSPAEDQTIQAPPVSVSALARDSPLTPNEMSSSTLTSPIEASWVSSQNDSPGDASEGPEYLAIGNLDPRGRTASCQSHSSNAESSSSNLFSSSSSQKPDSAASSLGDQEGGGESQLSSVLRRSSFSEGQTLTVTSGAKKSHIRSHSDTSIASRGAPESCNDKAKLRGPLPYSGQSSEVSTPSSLYMEYEGGRYLCSGEGMFRRPSEGQSLISYLSEQDFGSCADLEKENAHFSISESLIAAIELMKCNMMSQCLEEEEVEEEDSDREIQELKQKIRLRRQQIRTKNLLPMYQEAEHGSFRVTSSSSQFSSRDSAQLSDSGSADEVDEFEIQDADIRRNTASSSKSFVSSQSFSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMAIPSRVLRKWDFSKYYVSNFSKDLLIKIWNDPLFNVQDINSALYRKVKLLNQVRLLRVQLCHMKNMFKTCRLAKELLDSFDTVPGHLTEDLHLYSLNDLTATRKGELGPRLAELTRAGATHVERCMLCQAKGFICEFCQNEDDIIFPFELHKCRTCEECKACYHKACFKSGSCPRCERLQARREALARQSLESYLSDYEEEPAEALALEAAVLEAT

SEQ ID NO: 3 - protein, 570 aa, artificial
  synthetic
  MISC_FEATURE (1)..(570): Nox2
MGNWAVNEGLSIFVILVWLGLNVFLFVWYYRVYDIPPKFFYTRKLLGSALALARAPAACLNFNCMLILLPVCRNLLSFLRGSSACCSTRVRRQLDRNLTFHKMVAWMIALHSAIHTIAHLFNVEWCVNARVNNSDPYSVALSELGDRQNESYLNFARKRIKNPEGGLYLAVTLLAGITGVVITLCLILIITSSTKTIRRSYFEVFWYTHHLFVIFFIGLAIHGAERIVRGQTAESLAVHNITVCEQKISEWGKIKECPIPQFAGNPPMTWKWIVGPMFLYLCERLVRFWRSQQKVVITKVVTHPFKTIELQMKKKGFKMEVGQYIFVKCPKVSKLEWHPFTLTSAPEEDFFSIHIRIVGDWTEGLFNACGCDKQEFQDAWKLPKIAVDGPFGTASEDVFSYEVVMLVGAGIGVTPFASILKSVWYKYCNNATNLKLKKIYFYWLCRDTHAFEWFADLLQLLESQMQERNNAGFLSYNIYLTGWDESQANHFAVHHDEEKDVITGLKQKTLYGRPNWDNEFKTIASQHPNTRIGVFLCGPEALAETLSKQSISNSESGPRGVHFIFNKENF

SEQ ID NO: 4 - protein, 570 aa, Mus musculus
  MISC_FEATURE (1)..(570): Nox2_murine
MGNWAVNEGLSIFVILVWLGLNVFLFINYYKVYDDGPKYNYTRKLLGSALALARAPAACLNFNCMLILLPVCRNLLSFLRGSSACCSTRIRRQLDRNLTFHKMVAWMIALHTAIHTIAHLFNVEWCVNARVGISDRYSIALSDIGDNENEEYLNFAREKIKNPEGGLYVAVTRLAGITGIVITLCLILIITSSTKTIRRSYFEVFWYTHHLFVIFFIGLAIHGAERIVRGQTAESLEEHNLDICADKIEEWGKIKECPVPKFAGNPPMTWKWIVGPMFLYLCERLVRFWRSQQKVVITKVVTHPFKTIELQMKKKGFKMEVGQYIFVKCPKVSKLEWHPFTLTSAPEEDFFSIHIRIVGDWTEGLFNACGCDKQEFQDAWKLPKIAVDGPFGTASEDVFSYEVVMLVGAGIGVTPFASILKSVWYKYCDNATSLKLKKIYFYWLCRDTHAFEWFADLLQLLETQMQERNNANFLSYNIYLTGWDESQANHFAVHHDEEKDVITGLKQKTLYGRPNWDNEFKTIASEHPNTTIGVFLCGPEALAETLSKQSISNSESGPRGVHFIFNKENF

(19) United States
(12) Patent Application Publication
(10) Pub. No.: US 2019/0145961 A1
(43) Pub. Date: May 16, 2019

(54) PPAR AGONIST OR LXR AGONIST FOR USE IN THE TREATMENT OF SYSTEMIC LUPUS ERYTHEMATOSUS BY MODULATION OF LAP ACTIVITY

(71) Applicants: Washington University, Clayton, MO (US); St. Jude Children's Research Hospital

(72) Inventors: Herbert W. Virgin, Saint Louis, MO (US); Douglas R. Green, Memphis, TN (US); Jennifer Martinez, Research Triangle Park, NC (US)

(73) Assignees: Washington University, Clayton, MO (US); St. Jude Children's Research Hospital, Memphis, TN (US)

(21) Appl. No.: 16/095,666

(22) PCT Filed: Apr. 20, 2017
(86) PCT No.: PCT/IB2017/052284

§ 371 Date: Oct. 22, 2018

Related U.S. Application Data
(60) Provisional application No. 62/325,110, filed on Apr. 20, 2016.

(51) Int. Cl. G01N 33/50

(52) U.S. Cl. CPC G01N 33/5041; G01N 2333/523; G01N 2800/7095; G01N 2333/5412; G01N 2333/5428

(57) ABSTRACT

Compositions and methods are provided for modifying diagnosing and treating inflammatory disease. The methods and compositions can be used to ameliorate the effects of a deficiency in the LAP pathway for clearing dead cells. Thus, methods are further provided for modulating dead cell clearance using an effective amount of a pharmaceutical composition that targets the LAP pathway. Accordingly, pharmaceutical compositions that target the LAP pathway re provided herein. The methods and compositions described herein can be used to treat inflammatory disease, such as systemic lupus erythematosus (SLE).

### FIELD OF THE INVENTION

The invention relates to the field of cell biology and immunology. In particular, the invention relates to a method for modulating the LAP pathway in order to reduce inflammation in subjects. The methods and compositions can be used to treat symptoms of SLE and other inflammatory diseases in LAP-deficient subjects.

### REFERENCE TO A SEQUENCE LISTING SUBMITTED ELECTRONICALLY AS A TEXT FILE

The instant application contains a Sequence Listing which has been submitted in ASCII format via EFS-Web and is hereby incorporated by reference in its entirety. Said ASCII copy, created on Apr. 20, 2017, is named S88435_1150WO_0031_6_Seq_List.txt, and is 1.07 MB in size.

### BACKGROUND OF THE INVENTION

Macroautophagy (herein, autophagy) is a catabolic, cell survival mechanism activated during nutrient scarcity involving degradation and recycling of unnecessary or dysfunctional components. The proteins of autophagy machinery often interact with pathogens, such as Salmonella enterica, Listeria monocytogenes, Aspergillus fumigatus and Shigella flexneri, and function to quarantine and degrade invading organisms (xenophagy). LC3 (mammalian homologue of Atg8) is the most commonly monitored autophagy-related protein, and its lipidated form, LC3-II, is present on autophagosomes during canonical autophagy.

LC3-associated phagocytosis (LAP) is a process triggered following phagocytosis of particles that engage cell-surface receptors such as TLR1/2, TLR2/6, TLR4, TIM4 and FcR (refs 5_7), resulting in recruitment of some, but not all, members of the autophagic machinery to stimulus-containing phagosomes, facilitating rapid phagosome maturation, degradation of engulfed pathogens, and modulation of immune responses. LAP and autophagy have been shown to be functionally and mechanistically distinct processes. Whereas the autophagosome is a double-membrane structure, the LAP-engaged phagosome (LAPosome) is composed of a single membrane. Autophagy requires the activity of the pre-initiation complex, but LAP does not. However, LAP requires some autophagic components, such as the Class III PI(3)K complex7,11, and elements of the ubiquitylation-like, protein conjugation systems (ATG5, ATG7).

There remain significant gaps in our ability to differentiate LAP from canonical autophagy, in terms of molecular mechanisms and specificity. The Class III PI(3)K-associated protein, Rubicon, has been identified as required for LAP, yet non-essential for autophagy. Rubicon facilitates VPS34 activity and sustained PtdIns(3)P presence on LAPosomes and stabilizes the NOX2 complex for reactive oxygen species (ROS) production, both of which are critical for progression of LAP.

Defects in dying cell clearance are postulated to underlie the pathogenesis of systemic lupus erythematosus (SLE). Mice lacking molecules associated with dying cell clearance develop SLE-like disease², and phagocytes from SLE patients often display defective clearance and increased inflammatory cytokine production when exposed to dying cells in vitro. Genome-wide association studies have identified polymorphisms in atg5 and possibly atg7, involved in both canonical autophagy and LAP, as predisposition markers for SLE. However, there remains a need for understanding the in vivo consequences of LAP deficiency as a means for understanding progression of inflammatory diseases, in particular SLE-like diseases.

### SUMMARY OF THE INVENTION

Compositions and methods are provided for modifying diagnosing and treating inflammatory disease. The methods and compositions can be used to ameliorate the effects of a deficiency in the LAP pathway for clearing dead cells. Thus, methods are further provided for modulating dead cell clearance using an effective amount of a pharmaceutical composition that targets the LAP pathway. Accordingly, pharmaceutical compositions that target the LAP pathway are provided herein. The methods and compositions described herein can be used to treat inflammatory disease, such as systemic lupus erythematosus (SLE).

### BRIEF DESCRIPTION OF THE FIGURES

FIG. 1 depicts the results of treatment with PPAR agonists in LAP-deficient mice. FIG. 1A shows IL-10 production in Rubicon deficient mice following administration of PPARγ agonists Rosiglitazone (ROS, 20 or 60 μM) or Tesaglitazar (TES, 6 or 20 μM). FIG. 1B shows IL-10 production in LysM-Cre− ATG7f/f and LysM-Cre+ ATG7f/f mice following administration of PPARβ/δ agonist GW0742 (GW, 20 μM). FIG. 1C shows IL-10 production in Rubicon deficient mice following administration of LXR agonists T0901317 (T09, 6 or 20 μM) or 22(R)-hydroxycholesterol (22®-HC, 20 or 6 μM). Red boxes indicate increase of IL-10 production by LAP-deficient macrophages over NT (no treatment) conditions.

FIG. 2 shows that mice with LAP deficiencies display symptoms of autoinflammatory disorder. Wild-type and deficient littermates were co-housed and aged for 52 weeks. A. Weights. B. Anti-dsDNA antibodies (Total Ig). C-D. Anti-nuclear antigens (ANA, Total Ig) in animals aged 52 wks, C). Antibodies to autoantigens commonly associated with autoimmune and autoinflammatory disorders. 3 mice per genotype, normalized background signals (D). In all cases, Cre indicates LysM-Cre. Error bars represent standard deviation (*p<0.001). Animal numbers are provided in Supplemental Methods. Color scheme represents LAP-deficient, autophagy-deficient genotypes (green), autophagy-deficient, LAP-sufficient (red), and autophagy-sufficient, LAP-deficient (blue). Values for one cohort of TIM4+/+ and TIM4−/− animals are shown for comparison in all cases (black) in A and B.

FIG. 3 depicts results showing that mice with LAP deficiencies display kidney pathology. A-D. Appearance of kidneys of co-housed, 52 wk. animals. DAPI (blue), anti-IgG (red, A), anti-C1q (red, C). Mean fluorescent intensity (MFI) of anti-IgG (B) and anti-C1q (D) in glomeruli E. Serum creatinine. Animal numbers are provided in Supplemental Methods. Error bars represent standard deviation (*p<0.001, **p<0.05). For histological assessment, at least 15 glomeruli were evaluated for each genotype. Color scheme represents LAP-deficient, autophagy-deficient genotypes (green), autophagy-deficient, LAP-sufficient (red), and autophagy-sufficient, LAP-deficient (blue). Values for one cohort of TIM4+/+ and TIM4−/− animals are shown for comparison in all cases (black) in E.

FIG. 4 shows that mice with LAP deficiencies display defective clearance of engulfed, dying cells, resulting in increased production of pro-inflammatory cytokines. A-D. 1×107, PKH26-labeled UV-irradiated wild-type thymocytes were injected intravenously into indicated animals expressing GFP-LC3. (A, B) Apoptotic thymocytes in spleen, liver, and kidney of indicated animals measured by flow cytometry. (C, D) Indicated serum cytokines. Error bars represent standard deviation (n=4, *p<0.001, **p<0.05). E. 2×107, UV-irradiated wild-type thymocytes were injected intravenously six times over 8 weeks into indicated animals (aged 6 weeks). Serum anti-nuclear antibodies (ANA, Total Ig) and anti-dsDNA antibodies (Total Ig) are shown at 16 wks. Results are presented as ratio to average value prior to injection for each individual animal. Error bars represent standard error (n=4, **p<0.05). Cre indicates LysM-Cre. The color scheme represents LAP-deficient, autophagy-deficient genotypes (green), autophagy-deficient, LAP-sufficient (red), and autophagy-sufficient, LAP-deficient (blue).

FIG. 5 depicts that mice with LAP deficiencies display symptoms of an autoinflammatory disorder. (A-E). Indicated serum cytokines in co-housed 52 wk old animals. In all cases, Cre indicates LysM-Cre. Error bars represent standard deviation (*p<0.001). Numbers of animals are provided in Supplemental Methods. Color scheme represents LAP-deficient, autophagy-deficient genotypes (green), autophagy-deficient, LAP-sufficient (red), and autophagy-sufficient, LAP-deficient (blue). Values for one cohort of TIM4+/+ and TIM4−/− animals are shown for comparison in all cases (black) in A-E.

FIG. 6 demonstrates that LAP contributes to expression of PPARδ-regulated transcripts in macrophages. FIG. 6A. Rbcn+/+ and Rbcn−/− mice were injected intraperitoneal with 2.0×10⁷ apoptotic thymocytes and peritoneal macrophages were harvested by peritoneal wash 24 hours post stimulation. The expression of target genes was verified by real-time PCR. FIG. 6B. 106 bone marrow derived-macrophages from Rbcn+/+ and Rbcn−/− mice were stimulated with apoptotic thymocytes (1:10) in vitro and expression of target genes was verified 12 hours after stimulation by real-time PCR.

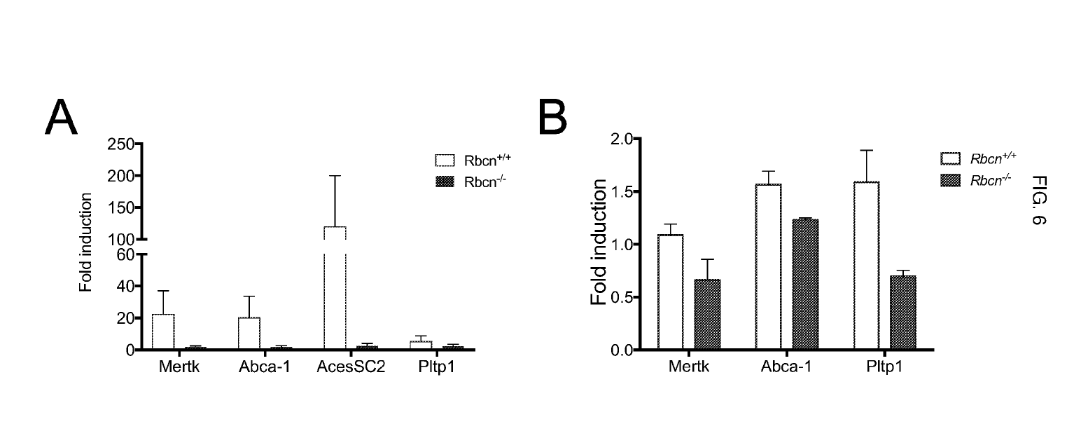

FIG. 7 shows that treatment with PPARδ agonist GW501516 reduces production of inflammatory cytokines in response to AT in vitro. 10⁶ bone marrow derived-macrophages from Rbcn+/+ and Rbcn−/− mice were stimulated with apoptotic thymocytes (1:10) in vitro for 18 hours, in the presence of GW501516 at different concentrations (+6 μM, ++20 μM, +++60 μM). Collected supernatants were assayed for cytokine production using Luminex technology.

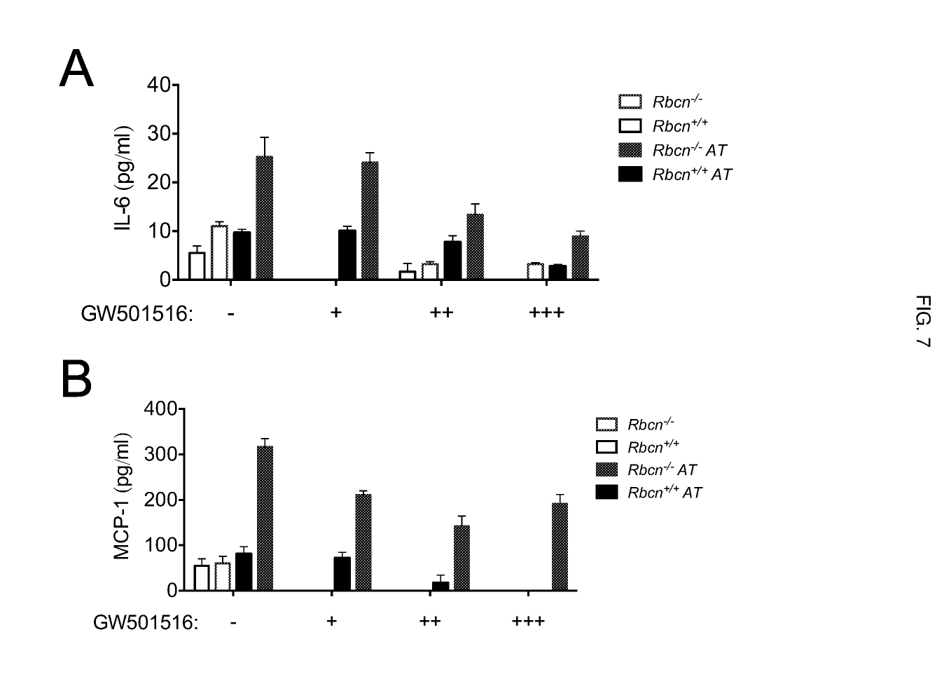

FIG. 8 demonstrates that treatment with PPARδ/b restores IL-10 production in response to apoptotic cells in LAP-deficient macrophages. 10⁶ bone marrow derived-macrophages from Rbcn+/+ and Rbcn−/− mice were stimulated with apoptotic thymocytes (1:10) in vitro for 18 hours, in the presence of GW501516 at different concentrations (++20 μM, +++60 μM). Collected supernatants were assayed for cytokine production using Luminex technology.

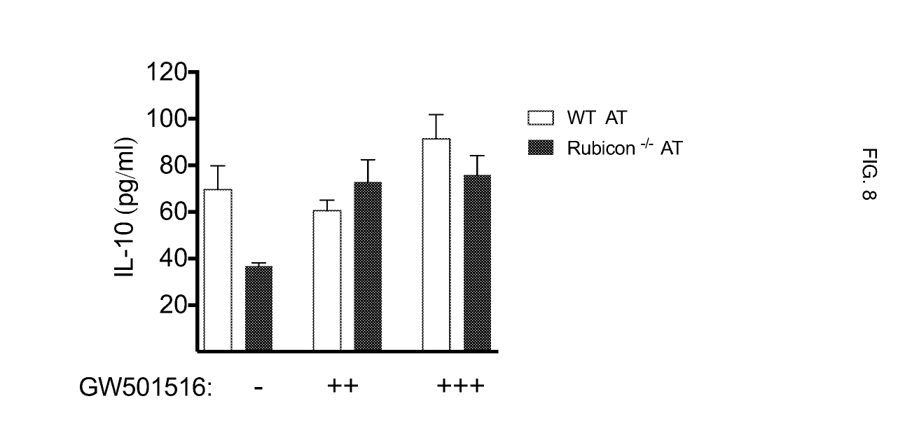

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### 1. Overview

Compositions and methods are provided herein for the treatment of conditions associated with a deficiency in the LC3-associated phagocytosis (LAP) pathway. LAP is a process in which some, but not all components of the autophagy machinery conjugate myosin associated light chain-3 (LC3) to phosphatidylethanolamine directly on the phagosome membrane, and the lipidated LC3 (LC3-II) then functions to facilitate lysosomal fusion and cargo destruction (e.g., LAP activity). Both LAP and canonical autophagy require ATG7, ATG3, ATG5, ATG12, and ATG16L for the process of LC3 lipidation. However, unlike canonical autophagy, LAP proceeds independently of the autophagic pre-initiation complex containing ULK1 and FIP200, and utilizes a distinct Beclin 1-VPS34 complex lacking ATG14. In contrast, LAP, but not canonical autophagy, requires NADPH oxidase-2 (NOX2), and Rubicon. These requirements for LAP and canonical autophagy can therefore distinguish between these two processes (See, Table 1). As used herein, the term “LAP-related” refers to any nucleic acid, protein, cytokine, or any other molecule that participates in the LAP pathway. LAP-related molecules include, but are not limited to Beclin1, BPS34, UVRAG, ATG7, ATG3, ATG5, ATG12, ATG16L, ATG3, ATG4, LC3 family LC3A, LC3B, GATE16, GABARAP), Rubicon, and NOX2. See, Table 1 for a description of selected LAP-related molecules and their associated function.

TABLE 1

| | | |
|---|---|---|
| Component | Function/Complex | Reference |

Components required for canonical autophagy and LAP

| | | |
|---|---|---|
| Beclin1 | Class III PI3K complex | 1, 2, 4, 6 |
| VPS34 | Class III PI3K complex | 1, 2, 4, 6 |
| UVRAG | Class III PI3K complex, vesicle sorting | 6 |
| ATG5 | ATG5-12-16L complex formation, | 1, 2, 4, 6 |
| | complex functions as E3 for LC3-II | |
| | generation | |
| ATG12 | ATG5-12-16L complex formation, | 6 |
| | complex functions as E3 for LC3-II | |
| | generation | |
| ATG16L | ATG5-12-16L complex formation, | 6 |
| | complex functions as E3 for LC3-II | |
| | generation | |
| ATG7 | ATG5-12-16L complex and LC3-PE | 1-6 |
| | formation (functions as E1) | |
| ATG3 | LC3-PE formation (function as E2) | 6 |
| ATG4 | LC3 processing | 6 |
| LC3 family (LC3A, | Maturation and fusion to lysosomal | 6 |
| LC3B, GATE16, | compartments | |
| GABARAP) | | |

Components required for canonical autophagy only

| | | |
|---|---|---|
| ULK1 | Pre-initiation complex | 2-6 |
| FIP200 | Pre-initiation complex | 4-6 |
| ATG13 | Pre-initiation complex | 4-6 |
| Ambra1 | Class III PI3K complex | 6 |
| WIPI2 | Recruitment of ATG5-12-16L | 6 |
| ATG14 | Class III PI3K complex | 6 |

Components required for LAP only

| | | |
|---|---|---|
| Rubicon | Localization and activity of Class | 6 |
| | III PI3K complex, stabilization of | |
| | NOX2 complex | |
| NOX2 | NADPH oxidase, ROS production, | 6 |
| | recruitment of ATG5-12-16L and | |
| | LC3 conjugation systems | |

1. Sanjuan, M. A. et al. Toll-like receptor signalling in macrophages links the autophagy pathway to phagocytosis. Nature 450, 1253-1257 (2007).
2. Martinez, J. et al. Microtubule-associated protein 1 light chain 3 alpha (LC3)-associated phagocytosis is required for the efficient clearance of dead cells. Proceedings of the National Academy of Sciences of the United States of America 108, 17396-17401, doi: 10.1073/pnas.1113421108 (2011).
3. Florey, O., Kim, S. E., Sandoval, C. P., Haynes, C. M. & Overholtzer, M. Autophagy machinery mediates macroendocytic processing and entotic cell death by targeting single membranes. Nat Cell Biol 13, 1335-1343, doi: 10.1038/ncb2363 (2011).
4. Henault, J. et al. Noncanonical autophagy is required for type I interferon secretion in response to DNA-immune complexes. Immunity 37, 986-997, doi: 10.1016/j.immuni.2012.09.014 (2012).
5. Kim, J. Y. et al. Noncanonical autophagy promotes the visual cycle. Cell 154, 365-376, doi: 10.1016/j.cell.2013.06.012 (2013).
6. Martinez, J. et al. Molecular characterization of LC3-associated phagocytosis (LAP) reveals distinct roles for Rubicon, NOX2, and autophagy proteins. Nature cell biology, 17, 893-906.

Accordingly, the term “LAP-deficient” refers to an alteration in the LAP pathway such that the LAP pathway does not function properly. That is, a LAP-deficient organism does not effectively clear the cargo of the phagocytes, including dead cells, without increased inflammation. A LAP-deficient subject could have an increase or decrease in the expression or activity of any LAP related molecule, or a defect in the subject's immune response to LAP-related dead cell clearance. Particularly, a LAP-deficient subject has in increase in pro-inflammatory cytokines or a decrease in anti-inflammatory cytokines (i.e., IL-10), which may lead to increased inflammation and symptoms of SLE.

### 2. Methods of Treatment

Methods and compositions are provided herein for decreasing inflammation in a subject comprising administration of an effective amount of a pharmaceutical composition that targets the LAP pathway. In certain embodiments the subject to be treated is a LAP-deficient subject, a subject with increased inflammation, or a subject with decreased dead cell clearance when compared to an appropriate control. In certain embodiments administration of an effective amount of a pharmaceutical composition that targets the LAP pathway can decrease the symptoms of LAP-deficiency, decrease inflammation, or increase dead cell clearance in a subject. “Treatment” is herein defined as curing, healing, alleviating, relieving, altering, remedying, ameliorating, improving, or affecting the condition or the symptoms of a LAP-deficient subject. The subject to be treated can be suffering from or at risk of developing an inflammatory disease or be at risk of developing any disease associated with LAP-deficiency. Reducing at least one symptom of a LAP-deficiency, inflammation, SLE, or decreased dead cell clearance refers to a statistically significant reduction of at least one symptom. Such decreases or reductions can include, for example, at least a 5%, 10%, 20%, 30%, 40%, 50%, 60%, 70%, 80%, 90%, 95%, or 100% decrease in the measured or observed level of at least one symptom, as disclosed elsewhere herein.

In some embodiments, the subject is a LAP-deficient subject having reduced expression of a LAP-related molecule. As used herein, the term “reduced” refers to any reduction in the expression or activity of a LAP-related molecule when compared to the corresponding expression or activity of the same LAP-related molecule in a control cell. Such a reduction may be up to 5%, 10%, 20%, 30%, 40%, 50%, 60%, 70%, 80%, 90%, 95%, or up to 100%. Accordingly, the term “reduced” encompasses both a partial knockdown and a complete knockdown of the activity of a LAP-related molecule.

By “subject” is intended animals. In specific embodiments, subjects are mammals, e.g., primates or humans. In other embodiments, subjects include domestic animals, such as a feline or canine, or agricultural animals, such as a ruminant, horse, swine, poultry, or sheep. In specific embodiments, the subject undergoing treatment with the pharmaceutical formulations of the invention is a human. In some embodiments, the human undergoing treatment can be a newborn, infant, toddler, preadolescent, adolescent or adult. The subjects of the invention may be suffering from the symptoms of an inflammatory disorder or may be at risk for developing an inflammatory disorder.

The methods and compositions disclosed herein a method of modulating LAP activity in a cell. In one embodiment, a method of increasing LAP activity in a cell comprises administering to the cell an effective amount of an agent which increases or enhances the biological activity of NOX2. In another embodiment, a method of increasing LAP activity in a cell comprises administering to the cell an effective amount of an agent which increases or enhances the biological activity of Rubicon. In one non-limiting embodiment, a method of decreasing LAP activity in a cell comprises administering to the cell an effective amount of an agent which decreases or inhibits the biological activity of Rubicon. In another non-limiting embodiment, a method of decreasing LAP activity in a cell comprises administering to the cell an effective amount of an agent which decreases or inhibits the biological activity of NOX2.

LAP activity can be determined by measuring dead cell clearance or by the methods disclosed herein in the Examples. One method to monitor LAP or LAP activity is to use Western blot analysis to identify key components such as Rubicon and LC3-II. Further, as disclosed elsewhere herein, LAP activity can be measured using immunofluorescence to identify LC3 associated with phagosomes, or flow cytometry. Any method known in the art can be used for measuring LAP activity, including those described in Martinez et al. (2015) Nature Cell Biology 17: 893-906, herein incorporated by reference in the entirety.

A. Inflammatory Disease

In some embodiments, inflammatory disorders associated with a LAP deficiency can be treated or prevented. Inflammatory diseases can arise where there is an inflammation of the body tissue. The term “inflammatory diseases” as used herein, includes, but are not limited to, local inflammatory responses and systemic inflammation. In specific embodiments, the inflammatory disorder to be treated is systemic lupus erythematosus (SLE) or lupus (including nephritis, non-renal, discoid, alopecia). Further disorders that could be treated or prevented by the methods and compositions described herein include, but are not limited to: arthritis (rheumatoid arthritis, juvenile rheumatoid arthritis, osteoarthritis, psoriatic arthritis), psoriasis, dermatitis including atopic dermatitis; chronic autoimmune urticaria, polymyositis/dermatomyositis, toxic epidermal necrolysis, systemic scleroderma and sclerosis, respiratory distress syndrome, adult respiratory distress syndrome (ARDS), meningitis, allergic rhinitis, encephalitis, uveitis, colitis, glomerulonephritis, allergic conditions, eczema, asthma, conditions involving infiltration of T cells and chronic inflammatory responses, atherosclerosis, autoimmune myocarditis, leukocyte adhesion deficiency, juvenile onset diabetes, multiple sclerosis, allergic encephalomyelitis, immune responses associated with acute and delayed hypersensitivity mediated by cytokines and T-lymphocytes, tuberculosis, sarcoidosis, granulomatosis including Wegener's granulomatosis, agranulocytosis, vasculitis (including ANCA), aplastic anemia, Coombs positive anemia, Diamond Blackfan anemia, immune hemolytic anemia including autoimmune hemolytic anemia (AIHA), pernicious anemia, pure red cell aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome, myasthenia gravis, antigen-antibody complex mediated diseases, anti-glomerular basement membrane disease, anti-phospholipid antibody syndrome, allergic neuritis, Bechet disease, Castleman's syndrome, Goodpasture's Syndrome, Lambert-Eaton Myasthenic Syndrome, Reynaud's syndrome, Sjorgen's syndrome, Stevens-Johnson syndrome, solid organ transplant rejection (including pretreatment for high panel reactive antibody titers, IgA deposit in tissues, etc), graft versus host disease (GVHD), pemphigoid bullous, pemphigus (all including vulgaris, foliaceus), autoimmune polyendocrinopathies, Reiter's disease, stiff-man syndrome, giant cell arteritis, immune complex nephritis, IgA nephropathy, IgM polyneuropathies or IgM mediated neuropathy, idiopathic thrombocytopenic purpura (ITP), thrombotic throbocytopenic purpura (TTP), autoimmune thrombocytopenia, autoimmune disease of the testis and ovary including autoimune orchitis and oophoritis, primary hypothyroidism; autoimmune endocrine diseases including autoimmune thyroiditis, chronic thyroiditis (Hashimoto's Thyroiditis), subacute thyroiditis, idiopathic hypothyroidism, Addison's disease, Grave's disease, autoimmune polyglandular syndromes (or polyglandular endocrinopathy syndromes), Type I diabetes also referred to as insulin-dependent diabetes mellitus (IDDM) and Sheehan's syndrome; autoimmune hepatitis, Lymphoid interstitial pneumonitis (HIV), bronchiolitis obliterans (non-transplant) vs NSIP, Guillain-Barre'Syndrome, Large Vessel Vasculitis (including Polymyalgia Rheumatica and Giant Cell (Takayasu's) Arteritis), Medium Vessel Vasculitis (includingKawasaki's Disease and Polyarteritis Nodosa), ankylosing spondylitis, Berger's Disease (IgA nephropathy), Rapidly Progressive Glomerulonephritis, Primary biliary cirrhosis, Celiac sprue (gluten enteropathy), Cryoglobulinemia, ALS, or coronary artery disease.

In one embodiment, the method of treating an inflammatory disease comprises administering to the subject a therapeutically effective amount of an agent which increases or enhances the biological activity of NOX2 or Rubicon. For example, the inflammatory disease can be an inflammatory disease associated with a defect in the LAP pathway and/or SLE.

In some embodiments, administration of an effective amount of a pharmaceutical composition that targets the LAP pathway results in an increase in anti-inflammatory cytokine production. As used herein, an “increase in” or “increasing” anti-inflammatory cytokine production comprises any statistically significant increase the anti-inflammatory cytokine level when compared to an appropriate control. Such increases can include, for example, at least a 5%, 10%, 20%, 30%, 40%, 50%, 60%, 70%, 80%, 90%, 100%, 150%, 200% or greater increase in the anti-inflammatory cytokine level. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in the anti-inflammatory cytokine level. Anti-inflammatory cytokines of the invention include interleukin (IL)-1 receptor antagonist, IL-4, IL-10, IL-11, and IL-13, IL-16, IFN-alpha, TGF-beta, G-CSF. Methods to assay for the level of anti-inflammatory cytokine level, are known. See, for example, Leng S., et al. (2008) J Gerontol A Biol Sci Med Sci 63(8): 879-884. Methods to assay for the production of anti-inflammatory cytokines include multiplex bead assay, ELISPOT and flow cytometry. See, for example, Maecker et al. (2005) BMC Immunology 6:13.

Methods and compositions also include those which decrease proinflammatory cytokine production, which may decrease or prevent an inflammatory response. As used herein, a decrease in the level of pro-inflammatory cytokine production comprises any statistically significant decrease in the level of pro-inflammatory cytokine production in a subject when compared to an appropriate control. Such decreases can include, for example, at least a 5%, 10%, 20%, 30%, 40%, 50%, 60%, 70%, 80%, 90%, or 100% decrease in the level of proinflammatory cytokines. Proinflammatory cytokines of the invention include IL1-alpha, IL1-beta, TNF-alpha, IL-2, IL-3, IL-6, IL-7, IL-9, IL-12, IL-17, IL-18, TNF-alpha, LT, LIF, Oncostatin, or IFN-alpha, IFN-beta, IFN-gamma. Methods to assay for cytokine levels are known and include, for example Leng S., et al. (2008) J Gerontol A Biol Sci Med Sci 63(8): 879-884. Methods to assay for the production of pro-inflammatory cytokines include multiplex bead assay, ELISPOT and flow cytometry. See, for example, Maecker et al. (2005) BMC Immunology 6:13.

Inflammatory cytokine production can also be measured by assaying the ratio of anti-inflammatory cytokine production to proinflammatory cytokine production. In specific aspects, the ratio of anti-inflammatory cytokine production to proinflammatory cytokine production is increased by about 1, 2, 3, 4, 5, 6, 7, 8, 9, 10, 20, 30, 40, 50, 60, 70, 80, 90, 100, 300, 600, 900, 1000 fold or greater when compared to an appropriate control. In other aspects, the ratio of anti-inflammatory cytokine production to pro-inflammatory cytokine production is increased by about 1 to 5 fold, about 5 to 10 fold, about 10 to 20 fold, about 20 to 30 fold, about 30 to 40 fold, about 40 fold to 60 fold, about 60 fold to 80 fold, about 80 fold to about 100 fold, about 100 to 200 fold, about 200 fold to 300 fold, about 300 to 400 fold, about 400 to about 500 fold, about 500 to about 500 fold, about 500 fold to about 700 fold, about 700 fold to 800 fold, about 800 fold to about 1000 fold or greater when compared to an appropriate control. Methods to determine the ratio of anti-inflammatory cytokine production to pro-inflammatory cytokine production can be found, for example, Leng S., et al. (2008) J Gerontol A Biol Sci Med Sci 63(8): 879-884. Methods to assay for the production of cytokines include multiplex bead assay, ELISPOT and flow cytometry. See, for example, Maecker et al. (2005) BMC Immunology 6:13.

B. Systemic Lupus Erythematosus (SLE)

In certain embodiments, administration of an effective amount of a pharmaceutical composition that targets the LAP pathway decreases the symptoms of SLE or lupus nephritis. Systemic lupus erythematosus (SLE) is a systemic autoimmune disease (or autoimmune connective tissue disease) that can affect any part of the body. As occurs in other autoimmune diseases, the immune system attacks the body's cells and tissue, resulting in inflammation and tissue damage. SLE can induce abnormalities in the adaptive and innate immune system, as well as mount Type III hypersensitivity reactions in which antibody-immune complexes precipitate and cause a further immune response. SLE most often damages the joints, skin, lungs, heart, blood components, blood vessels, kidneys, liver and nervous system. The course of the disease is unpredictable, often with periods of increased disease activity (called “flares”) alternating with suppressed or decreased disease activity. A flare has been defined as a measurable increase in disease activity in one or more organ systems involving new or worse clinical signs and symptoms and/or laboratory measurements. It must be considered clinically significant by the assessor and usually there would be at least consideration of a change or an increase in treatment (Ruperto et al., 2010). SLE can manifest as mild, moderate, or severe.

The most common causes of death in lupus patients include accelerated cardiovascular disease (likely associated with increased inflammation and perhaps additionally increased by select lupus therapies), complications from renal involvement and infections. SLE is one of several diseases known as “the great imitators” because it often mimics or is mistaken for other illnesses. SLE is a classical item in differential diagnosis, because SLE symptoms vary widely and come and go unpredictably. Diagnosis can thus be elusive, with some people suffering unexplained symptoms of untreated SLE for years. Common initial and chronic symptoms include fever, malaise, joint pains, myalgias, fatigue, and temporary loss of cognitive abilities. Because they are so often seen with other diseases, these signs and symptoms are not part of the American College of Rheumatology SLE classification criteria. When occurring in conjunction with other signs and symptoms, however, they are suggestive.

The most common clinical symptom which brings a patient for medical attention is joint pain, with the small joints of the hand and wrist usually affected, although nearly all joints are at risk. Unlike rheumatoid arthritis, many lupus arthritis patients will have joint swelling and pain, but no X-ray changes and minimal loss of function. SLE patients are at particular risk of developing articular tuberculosis. An association between osteoporosis and SLE has been found, and SLE may be associated with an increased risk of bone fractures in relatively young women.

Dermatological manifestations are common in subjects with SLE at some point in their disease, such as the classic malar rash (or butterfly rash). Subjects may exhibit chronic thick, annual scaly patches on the skin (referred to as discoid lupus). Alopecia, mouth ulcers, nasal ulcers, and photosensitive lesions on the skin are also possible manifestations, as well as anemia. Subjects with SLE may have an association with antiphospholipid antibody syndrome (a thrombotic disorder), wherein autoantibodies to phospholipids are present in their serum. Abnormalities associated with antiphospholipid antibody syndrome include a paradoxical prolonged partial thromboplastin time (which usually occurs in hemorrhagic disorders) and a positive test for antiphospholipid antibodies; the combination of such findings has earned the term “lupus anticoagulant-positive.” SLE patients with anti-phospholipid autoantibodies have more ACR classification criteria of the disease and may suffer from a more severe lupus phenotype.

A subject with SLE may have inflammation of various parts of the heart, such as pericarditis, myocarditis, and endocarditis. The endocarditis of SLE is characteristically noninfective (Libman-Sacks endocarditis), and involves either the mitral valve or the tricuspid valve. Atherosclerosis also tends to occur more often and advances more rapidly than in the general population. Lung and pleura inflammation can cause pleuritis, pleural effusion, lupus pneumonitis, chronic diffuse interstitial lung disease, pulmonary hypertension, pulmonary emboli, pulmonary hemorrhage, and shrinking lung syndrome. Painless hematuria or proteinuria may often be the only presenting renal symptom. Acute or chronic renal impairment may develop with lupus nephritis, leading to acute or end-stage renal failure. A histological hallmark of SLE is membranous glomerulonephritis with “wire loop” abnormalities. This finding is due to immune complex deposition along the glomerular basement membrane, leading to a typical granular appearance in immunofluorescence testing.

Neuropsychiatric syndromes can result when SLE affects the central or peripheral nervous systems. The American College of Rheumatology defines 19 neuropsychiatric syndromes in systemic lupus erythematosus. The diagnosis of neuropsychiatric syndromes concurrent with SLE is one of the most difficult challenges in medicine, because it can involve so many different patterns of symptoms, some of which may be mistaken for signs of infectious disease or stroke. The most common neuropsychiatric disorder people with SLE have is headache, although the existence of a specific lupus headache and the optimal approach to headache in SLE cases remains controversial. Other common neuropsychiatric manifestations of SLE include cognitive dysfunction, mood disorder (including depression), cerebrovascular disease, seizures, polyneuropathy, anxiety disorder, cerebritis, and psychosis. CNS lupus can rarely present with intracranial hypertension syndrome, characterized by an elevated intracranial pressure, papilledema, and headache with occasional abducens nerve paresis, absence of a space-occupying lesion or ventricular enlargement, and normal cerebrospinal fluid chemical and hematological constituents. More rare manifestations are acute confusional state, Guillain-Barre syndrome, aseptic meningitis, autonomic disorder, demyelinating syndrome, mononeuropathy (which might manifest as mononeuritis multiplex), movement disorder (more specifically, chorea), myasthenia gravis, myelopathy, cranial neuropathy and plexopathy. The neural manifestation of lupus is known as neuropsychiatric systemic lupus erythematosus (NPSLE). One aspect of this disease is severe damage to the epithelial cells of the blood-brain barrier.

Antinuclear antibody (ANA) testing, anti-dsDNA, and anti-extractable nuclear antigen (anti-ENA) responses form the mainstay of SLE serologic testing. Several techniques are used to detect ANAs (Lu et al., 2012; Bruner et al., 2012). Clinically the most widely used method is indirect immunofluorescence. The pattern of fluorescence suggests the type of antibody present in the patient's serum. Direct immunofluorescence can detect deposits of immunoglobulins and complement proteins in the patient's skin. When skin not exposed to the sun is tested, a positive direct IF (the so-called Lupus band test) is an evidence of systemic lupus erythematosus.

Deficiencies in the LAP pathway that result in failure of dead cell clearance can lead to an autoinflammatory response with lupus-like symptoms. Accordingly, administration of an effective amount of a pharmaceutical composition that targets the LAP pathway can restore the function of the pathway and decrease symptoms of SLE that result from deficiencies in the LAP pathway. Any symptom of SLE as described herein can be reduced by the methods described herein. In a particular embodiment, inflammation is reduced by administration of an effective amount of a pharmaceutical composition that targets the LAP pathway in a subject experiencing SLE symptoms.

C. Dead Cell Clearance

Multicellular organisms execute the majority of unwanted cell populations in a regulated fashion via the process of apoptosis. Examples of unwanted cells include excess cells generated during development, cells infected with intracellular bacteria or viruses, transformed or malignant cells capable of tumorigenesis, and cells irreparably damaged by cytotoxic agents. Swift removal of these cells is necessary for maintenance of overall health and homeostasis and prevention of autoimmunity, pathogen burden, or cancer. Quick removal of dying cells is a key final step, if not the ultimate goal of the apoptotic program. As described above, LAP plays an important role in the clearance of dead cells following engulfment, including the recruitment of cytokines.

LAP is triggered when an extracellular particle, such as a pathogen, immune complex, or dead cell, is sensed by an extracellular receptor, including Toll-like receptor1/2 (TLR1/2), TLR2/6, TLR4, FcR, and TIM4, and phagocytosed. This engulfment recruits some, but not all, members of the autophagy machinery to the cargo-containing vesicle. It is the activity of these autophagic players that facilitates the rapid processing of the cargo via fusion with the lysosomal pathway, which can have a critical role in the degradation of engulfed cargo, as well as modulate the resulting immune response. Despite sharing common molecular machinery, there currently exist several distinctions that differentiate LAP from canonical autophagy. Originally, LAP and autophagy were distinguished by the structure of the LC3-decorated phagosome (or LAPosome) and the rapidity with which LAP occurs. EM analysis revealed that LAP results in single-membrane structures, as opposed to the double-membrane autophagosomes surrounding autophagic cargo. Whereas LC3-decorated autophagosomes can take hours to form, LC3-II can be detected on LAPosomes in as few as 10 min after phagocytosis, and phosphatidylinositol 3-phosphate (PI(3)P) activity can be seen at the LAPosome within minutes after phagocytosis.

Although a majority of the core autophagy components are required for LAP, there exist some critical differences that can distinguish the two processes. Under basal conditions, mTOR inhibits the pre-initiation complex, comprised of FIP200, autophagy-related gene13 (ATG13), and ULK1/2, and hence autophagy. However, the pre-initiation complex is dispensable for LAP. Furthermore, canonical autophagy requires the ULK1-dependent release of a Beclin1-activating cofactor, Ambra1, from the dynein motor complex, and the function of WIPI2, whereas LAP does not.

Both LAP and canonical autophagy require the class III PI3K complex, which contains the core components Beclin1, VPS34, and VPS15. It can, however, differ in its additional composition. ATG14 and UVRAG are mutually exclusive in their association with the class III PI3K complex during autophagy, and silencing of either ATG14 or UVRAG inhibits canonical autophagy. LAP, on the other hand, only requires the activity of the UVRAG-containing class III PI3K complex, whereas ATG14 is dispensible.

Rubicon (RUN domain protein as Beclin 1 interacting and cysteine-rich containing) is a protein that associates constitutively with the UVRAG-containing class III PI3K complex. Rubicon is a negative regulator of autophagy (via its inhibition of VPS34 or by blocking GTPase Rab7 activation), and silencing of Rubicon results in an increase in the number of autophagosomes. During LAP, Rubicon is uniquely associated with LAPosomes (but not conventional phagosomes), and Rubicon-deficient cells are completely defective in LAP. Thus, Rubicon is a molecule that is uniquely required for LAP, but dispensable for canonical autophagy.

Studies suggest that the role for Rubicon in LAP is twofold. First, Rubicon promotes the association of the active class III PI3K complex with the LAPosome, thereby aiding in the localization of VPS34-mediated PI(3)P at the LAPosome. In both canonical autophagy and LAP, PI(3)P is required for the recruitment of the downstream ubiquitin-like conjugation systems, the ATG5-12 and LC3-PE conjugation systems. In LAP, Rubicon and PI(3)P have an additional role. Rubicon stabilizes NOX2, the predominant NADPH oxidase in phagocytes, by interacting with its p22phox subunit via its serine-rich domain (aa 567-625), a domain separate from the CCD domain (aa 515-550) responsible for its interaction with Beclin1 and the RUN domain (aa 49-180) responsible for its interaction with VPS34. Moreover, PI(3)P binds and stabilizes the p40phox subunit of NOX2. Collectively, Rubicon promotes the association of the active class III PI3K complex with the LAPosome and the production of PI(3)P (i.e., Rubicon activity). Rubicon and PI(3)P stabilize the active NOX2 complex to promote optimal reactive oxygen species (ROS) production, which is also required for successful LAP. Indeed, NOX2-deficeint cells fail to undergo LAP and scavenging of ROS by antioxidants, such as resveratrol, Tiron, or alpha-tocopherol is also an effective way to inhibit LAP.

In specific embodiments, administration of an effective amount of a pharmaceutical composition that targets the LAP pathway decreases the symptoms of a deficiency in dead cell clearance. Accordingly administration of an effective amount of a pharmaceutical composition that targets the LAP pathway can increase dead cell clearance. In certain embodiments, clearance of dead cells is increased because of a restoration of all or a portion of the LAP pathway. Methods for measuring dead cell clearance are known in the art and disclosed elsewhere herein.

### 3. Pharmaceutical Composition

The methods and compositions disclosed herein encompass administration of an effective amount of a pharmaceutical composition that targets the LAP pathway. A composition or molecule that targets the LAP pathway could be any molecule that increases or decreases (i.e., modulates) LAP activity. As used herein, the term “specifically” means the ability of a molecule that targets the LAP pathway to increase or decrease LAP activity without impacting other related processes (i.e., canonical autophagy). A molecule that targets the LAP pathway preferentially, increases or decreases LAP activity, but might impact other phagocytosis-related pathways. Accordingly, a molecule that targets the LAP pathway could be any LAP-related nucleic acid, protein, or cytokine, such as Beclin1, VPS34, UVRAG, ATG5, ATG12, ATG16L, ATG7, ATG3, ATG4, LC3A, LC3B, GATE16, GABARAP, Rubicon, or NOX2.

For example, various embodiments of the present invention pertain to methods for modulating LAP activity which comprise administering to a cell an effective amount of an agent which increases or enhances the biological activity of Rubicon and/or NOX2. An agent that increases or enhances the activity of Rubicon and/or NOX2 includes, but is not limited to, Rubicon and/or NOX2 itself, a functional agonistic fragment thereof, a Rubicon and/or NOX2 mimetic compound, a therapeutic vector which comprises a nucleic acid molecule encoding Rubicon protein, and a binding enhancer which enhances or prolongs the binding between Rubicon and the active class III PI3K complex with the LAPosome and between Rubicon and the active NOX2 complex.

Other non-limiting embodiments pertain to methods of increasing LAP activity or increasing dead cell clearance in a cell which comprise administering to the cell an effective amount of an agent which decreases or inhibits the biological activity of Rubicon and/or NOX2. An agent that decreases or inhibits the biological activity of Rubicon and/or NOX2 includes, but is not limited to, a functional antagonistic fragment of Rubicon and/or NOX2, an anti-Rubicon and/or anti-NOX2 antibody or fragment thereof such as an intrabody, another agent which inhibits or blocks Rubicon and/or NOX2 biological activity, or a nucleic acid targeted to the Rubicon and/or NOX2 gene, such as an antisense nucleic acid, a DNA construct for expression of an antisense RNA, a ribozyme, a DNA construct for expression of a ribozyme, a DNAzyme; or an RNAi.

The full-length amino acid sequence of murine Rubicon (GenBank accession number: AAH67390; gi145708948) has 941 amino acids, is designated SEQ ID NO: 1.

The full-length amino acid sequence of human Rubicon has 972 amino acids is designated SEQ ID NO: 2. (SEQ ID NO: 2)

Rubicon protein is predicted to comprise a conserved RUN domain, near the N-terminus, a cysteine-rich domain at the C-terminus, and a coiled-coil domain (CCD) or motif in the central region. The predicted CCD of murine Rubicon has a sequence of amino acid sequences 488 to 508 of SEQ ID NO: 1. The predicted CCD of human Rubicon has a sequence of amino acid sequences 518 to 538 of SEQ ID NO: 2. One of ordinary skill in the art would understand how to generate a Rubicon polypeptide in view of the disclosure of SEQ ID NO: 1 and SEQ ID NO: 2 using any of a number of experimental methods well-known to those of skill in the art. In one embodiment, a Rubicon polypeptide having biological activity of a native Rubicon protein, the biological activity of a native Rubicon protein is as described in the examples, including, but not limited to, promoting the association of the active class III PI3K complex with the LAPosome and the production of PI(3)P (i.e., Rubicon activity) and stabilization of the active NOX2 complex to promote optimal ROS production.

The NADPH Oxidase (nicotinamide adenine dinucleotide phosphate-oxidase, Nox) family of enzymes emerged during the evolutionary transition from unicellular to multicellular organisms and catalyze the reduction of oxygen to superoxide. Nox2 is a member of the Nox family and is known by a variety of aliases, including CYBB (Cytochrome b-245, beta polypeptide (chronic granulomatous disease)). Aliases of Nox2 include: CYBB, AMCBX2; CGD; GP91-1; GP91-PHOX; GP91PHOX; and p91-PHOX. An exemplary amino acid sequence of Nox2 is provided in GenBank Accession No. NM_000397.3 (SEQ ID NO: 3) and GenBank Accession No. NP_031833.3 (SEQ ID NO: 4, murine Nox2). Nox2 is also referred to as the phagocytic “respiratory burst oxidase” for its role in the innate immune response, specifically in phagocyte killing of ingested microbes

Members of the peroxisome proliferator-activated receptor γ/δ (PPARγ/δ) and liver X receptor (LXR) families, both important regulators of cellular lipid homeostasis, are activated during efferocytosis, and results in a positive feedback signal wherein the phagocytic receptors, such as members of the TAM family, are upregulated. Furthermore, cholesterol efflux machinery, such as 12-transmembrane protein ABCA1 (ATP-binding cassette sub-family A, member 1), is upregulated to accommodate the increase in cholesterol load. In some embodiments, the pharmaceutical composition that targets the LAP pathway is a PPAR agonist (e.g., a PPAR-α, PPAR-β/δ, or a PPAR-γ agonist), an LXR agonist (e.g., an LXR-α or LXR-β agonist), an RXR agonist (e.g., an RXR-α, RXR-β, or an RXR-γ agonist), an HNF-4 agonist, or a sirtuin-activating compound.

In methods of the invention wherein a PPAR agonist is administered to target the LAP pathway, the PPAR agonist may be any suitable PPAR agonist including, but not limited to, GW409544, LY-518674, LY-510929, TZD18, LTB4, oleylethanolamide, LY-465608, pirinixic acid, fatty acids (e.g., docohexaenoic acid, arachidonic acid, linoleic acid, C6-C18 fatty acid, and eicosatetraynoic acid), ragaglitazar, AD-5061, fenofibric acid, GW7647, GW9578, TAK-559, KRP-297/MK-0767, eicosatetraenoic acid, farglitazar, reglitazar, DRF 2519, pristanic acid, bezafibrate, clofibrate, 8S-hydroxyeicosatetraenoic acid, GW2331, NS-220, pterostilbene, tetradecylglycidic acid, ortylthiopropionic acid, WY14643, ciprofibrate, gemfibrozil, muraglitazar, tesaglitazar, eicosanoids (e.g., 15d-PGD₂, PGD₂, protacyclin, PGI₂, PGA.sub.1/2, PGB₂, 8-hydroxyeicosapentaienoic acid, 8-(R)hydroxyeicosatetraenoic acid, 8-(S)hydroxyeicosatetraenoic acid, 12-hydroxyeicosatetraenoic acid, LTB.sub.4, 9-(R/S)hydroxyoctadecadienoic acid, 13-(R/S)hydroxyoctadecadienoic acid, 20,8,9-hydroxyepoxyeicosatrienoic acid, 20,11,12-hydroxyepoxyeicosatrienoic acid, and 20,14,15-hydroxyepoxyeicosatrienoic acid), GW0742X, GW2433, GW9578, GW0742, L-783483, GW501516, retinoic acid, L-796449, L-165461, L-165041, SB-219994, LY-510929, AD-5061, L-764406, GW0072, nTzDpa, troglitazone, LY-465608, pioglitazone, SB-219993, 5-aminosalicyclic acid, GW1929, L-796449, GW7845,2-cyano-3,12-dioxooleana-1,9-dien-28-oic acid, L-783483, L-165461, AD5075, fluorenylmethoxycarbonyl-L-leucine, CS-045, indomethacin, rosiglitazone (BRL49653), SB-236636, GW2331, PAT5A, MCC555, bisphenol A diglycidyl ether, GW409544, GW9578, TAK-559, reglitazar, GW9578, ciglitazone, DRF2519, LG10074, ibuprofen, diclofenac, fenofibrate, naviglitazar, or pharmaceutically acceptable salts thereof. In specific embodiments the PPAR-γ agonist is Rosiglitazone. In other embodiments, the PPAR-β/δ agonist is GW0742.

In methods of disclosed herein, wherein an LXR is administered to target the LAP pathway, the LXR agonist may be any suitable LXR agonist including, but are not limited to tesaglitazar, TO901317, GW3965, T1317, acetyl-podocarpic dimer (APD), or pharmaceutically acceptable salts thereof. Other examples of LXR agonists suitable for said administration may be found in US Patent Application No. 2006/0205819 and references cited therein. In methods of disclosed herein, wherein an HNF-4 is administered to target the LAP pathway, the HNF-4 agonist may be any suitable HNF-4 agonist. In specific embodiments the LXR agonist is Tesaglitazar.

In methods of disclosed herein, wherein an RXR is administered to target the LAP pathway, the RXR agonist may be any suitable RXR agonist including, but are not limited to LG 100268 (i.e. 2-[1-(3,5,5,8,8-pentamethyl-5,6,7,8-tetrahydro-2-naphthyl)-cyclopropyl]-pyridine-5-carboxylic acid), LGD 1069 (i.e. 4-[(3,5,5,8,8-pentamethyl-5,6,7,8-tetrahydro-2-naphthyl)-2-carbonyl]-benzoic acid), AGN 194204,9-cis-retinoic acid, AGN 191701, bexarotene, BMS 649, and analogs, derivatives and pharmaceutically acceptable salts thereof. The structures and syntheses of LG 100268 and LGD 1069 are disclosed in Boehm, et al. J. Med. Chem. 38(16):3146-3155, 1994, incorporated by reference herein.

The pharmaceutical composition may be a liquid formulation or a solid formulation. When the pharmaceutical composition is a solid formulation it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip or a film. When the pharmaceutical composition is a liquid formulation it may be formulated as an oral solution, a suspension, an emulsion or syrup. Said composition may further comprise a carrier material independently selected from, but not limited to, the group consisting of lactic acid fermented foods, fermented dairy products, resistant starch, dietary fibers, carbohydrates, proteins, and glycosylated proteins. As used herein, the pharmaceutical composition could be formulated as a food composition, a dietary supplement, a functional food, a medical food, or a nutritional product as long as the required effect is achieved.

The pharmaceutical composition according to the invention, used according to the invention or produced according to the invention may also comprise other substances, such as an inert vehicle, or pharmaceutical acceptable adjuvants, carriers, preservatives etc., which are well known. By “therapeutically effective dose,” “therapeutically effective amount,” or “effective amount” is intended an amount of the composition or molecule that targets the LAP pathway that brings about a positive therapeutic response with respect to treatment or prevention. “Positive therapeutic response” refers to, for example, improving the condition of at least one of the symptoms of an inflammatory disorder, decreasing at least one symptom of SLE, and/or increasing dead cell clearance.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, one or both of the agents is infused over a period of less than about 4 hours, 3 hours, 2 hours or 1 hour. In still other embodiments, the infusion occurs slowly at first and then is increased over time.

Generally, the dosage of the composition that targets the LAP pathway will vary depending upon such factors as the patient's age, weight, height, sex, general medical condition and previous medical history. In specific embodiments, it may be desirable to administer the composition that targets the LAP pathway in the range of from about 1 to 100 mg/kg, 20 to 30 mg/kg, 30 to 40 mg/kg, 40 to 50 mg/kg, 50 to 60 mg/kg, 60 to 70 mg/kg, 70 to 80 mg/kg, 80 to 100 mg/kg, 5 to 10 mg/kg, 2 to 10 mg/kg, 10 to 20 mg/kg, 5 to 15 mg/kg, 1 to 10 mg/kg, 1 to 5 mg/kg, 2 to 5 mg/kg or any range in between 1 and 100 mg/kg.

In some embodiments of the invention, the method comprises administration of multiple doses of the composition that targets the LAP pathway. The method may comprise administration of 1, 2, 3, 4, 5, 6, 7, 8, 9, 10, 15, 20, 25, 30, 35, 40, or more therapeutically effective doses of a composition that targets the LAP pathway. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to improve the condition of at least one of the symptoms of an inflammatory disorder, decrease at least one symptom of SLE, and/or increase dead cell clearance. Changes in dosage may result and become apparent from the results of diagnostic assays for detecting inflammation, SLE symptoms, and dead cell clearance known in the art and described herein.

### 4. Detection of Expression

The methods disclosed herein include using LAP-related molecules to diagnose LAP-related disease states, such as inflammation, SLE, and failed dead cell clearance. In one embodiment, the method of evaluating expression of LAP-related molecules comprises detecting an NOX2 or Rubicon polypeptide in a biological sample. In another embodiment, the method of evaluating expression comprises detecting the amount of NOX2 or Rubicon mRNA in the biological sample. The term “biological sample” is intended to mean any biological sample obtained from an individual subject, including but not limited to a body fluid or a tissue sample, cell line, tissue culture, etc. Examples of body fluids include blood, semen, serum, plasma, urine, synovial fluid and spinal fluid.

In one non-limiting embodiment, the expression of NOX2 correlates to LAP activity. For example, increased expression of NOX2 indicates increased LAP activity, and decreased expression of NOX2 indicates decreased LAP activity. In one embodiment, the expression of Rubicon correlates to LAP activity. For example, increased expression of Rubicon indicates increased LAP activity, and decreased expression of Rubicon indicates decreased LAP activity. In another embodiment, the expression of Rubicon and NOX correlate individually to dead cell clearance. For example, increased expression of Rubicon or NOX2 indicates increased dead cell clearance.

In some embodiments, the method of evaluating expression of NOX2 or Rubicon comprises detecting an NOX2 or Rubicon polypeptide in the biological sample, which method comprises (a) contacting the biological sample with an anti-NOX2 or anti-Rubicon antibody or antigen binding portion thereof and (b) detecting the presence of an anti-NOX2 or anti-Rubicon antibody or the antigen binding portion thereof that is specifically bound to NOX2 or Rubicon polypeptide from the biological sample. The methods include, but are not limited to, Enzyme-Linked ImmunoSorbent Assay (ELISA), a Western blot, labeling the NOX2 polypeptide and identifying the labeled NOX2 polypeptide, a mass spectrometry, a gel electrophoresis, and a combination thereof. In one embodiment, the method of evaluating expression comprises detecting the amount of NOX2 or Rubicon mRNA in the biological sample. The methods include, but are not limited to a reverse transcription-polymerase chain reaction, Northern blotting, microarray, or a combination thereof.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decreases production. Molecules (e.g., transcription factors) that are involved in up-regulation or down-regulation are often called “activators” and “repressors,” respectively.

One agent useful for detecting NOX2 or Rubicon polypeptide is an antibody capable of binding to NOX2 or Rubicon polypeptide, preferably an antibody with a detectable label. Antibodies can be polyclonal, or more preferably, monoclonal. An intact antibody, or a fragment thereof (e.g., Fab or F(ab′).sub.2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The detection method of the invention can be used to detect NOX2 or Rubicon activity in a biological sample in vitro as well as in vivo. In vitro techniques for detection of NOX2 or Rubicon polypeptide include, but are not limited to, enzyme linked immunosorbent assay (ELISA), Western blot, labeling the ATG14L or Rubicon polypeptide and identifying the labeled NOX2 or Rubicon polypeptide using a technique such as immunofluorescence, mass spectrometry, gel electrophoresis, or immunoprecipitation. For a detailed explanation of methods for carrying out Western blot analysis (Sambrook et al., Molecular Cloning, A Laboratory Manual (2nd ed. 1989) at Chapter 18). The protein detection and isolation methods employed herein may also be such as those described in for example, Harlow, E. and Lane, D., 1988, “Antibodies: A Laboratory Manual,” Cold Spring Harbor Laboratory Press, Cold Spring Harbor, N.Y.

Detection of NOX2 or Rubicon activity can be accomplished, for example, by immunofluorescence techniques employing a fluorescently labeled antibody coupled with light microscopic, flow cytometric, or fluorimetric detection. Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a gamma counter or a scintillation counter or by autoradiography.

Methods for evaluating gene expression by detecting the amount of mRNA level in a cell, often but not always hybridization based, include, e.g., northern blots; dot blots; primer extension; nuclease protection; subtractive hybridization and isolation of non-duplexed molecules using, e.g., hydroxyapatite; solution hybridization; filter hybridization; amplification techniques such as RT-PCR and other PCR-related techniques such as differential display, LCR, AFLP, RAP, etc. (see, e.g., U.S. Pat. Nos. 4,683,195 and 4,683,202; PCR Protocols: A Guide to Methods and Applications (Innis et al., eds, 1990); Liang and Pardee, Science 257:967-971 (1992); Hubank & Schatz, Nuc. Acids Res. 22:5640-5648 (1994); Perucho et al., Methods Enzymol. 254:275-290 (1995)), fingerprinting, e.g., with restriction endonucleases (Ivanova et al., Nuc. Acids. Res. 23:2954-2958 (1995); Kato, Nuc. Acids Res. 23:3685-3690 (1995); and Shimkets et al., Nature Biotechnology 17:798-803, see also U.S. Pat. No. 5,871,697)); and the use of structure specific endonucleases (see, e.g., De Francesco, The Scientist 12:16 (1998)).

Nucleotide probes can be used to detect expression of a gene corresponding to the provided polynucleotide. In Northern blots, mRNA is separated electrophoretically and contacted with a probe. A probe is detected as hybridizing to an mRNA species of a particular size. The amount of hybridization can be quantified to determine relative amounts of expression. Probes can be used for in situ hybridization to cells to detect expression. Probes can also be used in vivo for diagnostic detection of hybridizing sequences. Probes can be labeled with a radioactive isotope or other types of detectable labels, e.g., chromophores, fluorophores and/or enzymes. Other examples of nucleotide hybridization assays are described in WO92/02526 and U.S. Pat. No. 5,124,246.

PCR is another means for detecting small amounts of target nucleic acids (see, e.g., Mullis et al., Meth. Enzymol. (1987) 155:335; U.S. Pat. Nos. 4,683,195; and 4,683,202). Two primer oligonucleotides that hybridize with the target nucleic acids can be used to prime the reaction. The primers can be composed of sequence within or 3′ and 5′ to the polynucleotides described herein. After amplification of the target by standard PCR methods, the amplified target nucleic acids can be detected by methods known in the art, e.g., Southern blot. mRNA or cDNA can also be detected by traditional blotting techniques (e.g., Southern blot, Northern blot, etc.) described in Sambrook et al., “Molecular Cloning: A Laboratory Manual” (New York, Cold Spring Harbor Laboratory, 1989) (e.g., without PCR amplification). In general, mRNA or cDNA generated from mRNA using a polymerase enzyme can be purified and separated using gel electrophoresis, and transferred to a solid support, such as nitrocellulose. The solid support can be exposed to a labeled probe and washed to remove any unhybridized probe. Duplexes containing the labeled probe can then be detected.

The terms “reverse transcription polymerase chain reaction” and “RT-PCR” refer to a method for reverse transcription of an RNA sequence to generate a mixture of cDNA sequences, followed by increasing the concentration of a desired segment of the transcribed cDNA sequences in the mixture without cloning or purification. Typically, RNA is reverse transcribed using a single primer (e.g., an oligo-dT primer) prior to PCR amplification of the desired segment of the transcribed DNA using two primers.

Techniques are available to expedite expression analysis and sequencing of large numbers of nucleic acids samples. For example, nucleic acid arrays have been developed for high density and high throughput expression analysis (see, e.g., Granjeuad et al., BioEssays 21:781-790 (1999); Lockhart & Winzeler, Nature 405:827-836 (2000)). Nucleic acid arrays refer to large numbers (e.g., hundreds, thousands, tens of thousands, or more) of nucleic acid probes bound to solid substrates, such as nylon, glass, or silicon wafers (see, e.g., Fodor et al., Science 251:767-773 (1991); Brown & Botstein, Nature Genet. 21:33-37 (1999); Eberwine, Biotechniques 20:584-591 (1996)). A single array can contain, e.g., probes corresponding to an entire genome, or to all genes expressed by the genome. The probes on the array can be DNA oligonucleotide arrays (e.g., GeneChip™, see, e.g., Lipshutz et al., Nat. Genet. 21:20-24 (1999)), mRNA arrays, cDNA arrays, EST arrays, or optically encoded arrays on fiber optic bundles (e.g., BeadArray™). The samples applied to the arrays for expression analysis can be, e.g., PCR products, cDNA, mRNA, etc.

As used herein, the term “microarray” refers to analysis of individual recombinant clones (e.g., cosmid, YAC, BAC, plasmid or other vectors) that are placed on a two-dimensional solid support (e.g., microscope slide). Each primary clone can be identified on the support by virtue of its location (row and column) on the solid support. Arrayed libraries of clones can be screened with RNA obtained from a specimen of interest upon conjugation of a fluorochrome.

Polypeptides described herein may be isolated and purified natural products, or may be produced partially or wholly using recombinant chemical synthesis techniques. “Peptide mimetics” or “peptidomimetics” are described in Fauchere, J. (1986) Adv. Drug Res. 15:29; Veber and Freidinger (1985) TINS p. 392; and Evans et al. (1987) J. Med. Chem. 30:1229. Peptide mimetics that are structurally similar to therapeutically useful peptides may be used to produce an equivalent therapeutic effect. Peptide mimetics may have significant advantages over polypeptide embodiments, including, for example: more economical production; greater chemical stability; enhanced pharmacological properties (half-life, absorption, potency, efficacy, etc.); altered specificity (e.g., a broad-spectrum of biological activities); reduced antigenicity; and others.

NOX2 or Rubicon protein variants can be generated through various techniques known in the art. For example, functional antagonistic fragments of the protein can be generated which are able to inhibit the function of the naturally occurring form of the protein, such as by competitively binding to another molecule that interacts with NOX2 or Rubicon protein. In addition, functional agonistic forms of the protein may be generated that constitutively express on or more NOX2 or Rubicon functional activities. Whether a change in the amino acid sequence of a peptide results in an NOX2 or Rubicon protein variant having one or more functional activities of a native NOX2 or Rubicon protein can be readily determined by testing the variant for a native NOX2 or Rubicon protein functional activity. As used herein, a “binding enhancer” refers to a compound capable of enhancing the binding between two binding partners when added to a reaction solution. Non-limiting examples of binding enhancers include compounds such as glutaraldehyde or carbodiimide.

The concentration of a binding enhancer in a reaction solution may be appropriately set according to the type of binding enhancer. More specifically, in the case of glutaraldehyde, for example, the final concentration in a reaction solution is typically from 0.1 to 25%, and preferably from 0.2 to 18%. The binding enhancer may be added to a reaction solution containing a conjugate of binding partners before diluting the reaction solution. The reaction solution to which a binding enhancer has been added can be diluted after incubation at 37° C. for several seconds to about 20 seconds, preferably two to ten seconds, or two to five seconds. When glutaraldehyde or carbodiimide is used as a binding enhancer, the reaction solution may be diluted immediately after the addition.

In one embodiment, nucleic acids comprising sequences encoding NOX2 or Rubicon protein, are administered to treat, inhibit, or prevent a disease or disorder associated with aberrant expression and/or activity of the LAP pathway, by way of gene therapy. In this embodiment, the nucleic acids produce their encoded protein that mediates a therapeutic effect.

In a certain embodiment, the compound comprises an expression cassette comprising nucleic acid sequences encoding an NOX2 or Rubicon polypeptide or functional fragment thereof, that express the NOX2 or Rubicon polypeptide or functional fragments thereof in a suitable host. In particular, such nucleic acid sequences have promoters operably linked to the NOX2 or Rubicon coding region, said promoter being inducible or constitutive, and, optionally, tissue-specific. Delivery of nucleic acid into a subject or cell may be either direct, in which case the subject or cell is directly exposed to the nucleic acid or nucleic acid-carrying vectors, or indirect, in which case, cells are first transformed with the nucleic acids in vitro, and then transplanted into the patient.

The nucleic acid may be directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. (1987); 262:4429-4432) (which can be used to target cell types specifically expressing the receptors), etc. The nucleic acid-ligand complexes can also be formed in which the ligand comprises a fusogenic viral peptide to disrupt endosomes, allowing the nucleic acid to avoid lysosomal degradation. In addition, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA (1989); 86:8932-8935; Zijlstra et al., Nature (1989); 342:435-438).

In a specific embodiment, a viral vector that contains nucleic acid encoding an NOX2 or Rubicon polypeptide or a functional fragment thereof may be used. For example, a retroviral vector can be used (see Miller et al., Meth. Enzymol. (1993); 217:581-599). These retroviral vectors contain the components necessary for the correct packaging of the viral genome and integration into the host cell DNA. More detail about retroviral vectors can be found in Boesen et al., Biotherapy (1994); 6:291-302, which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. (1994); 93:644-651; Kiem et al., Blood (1994); 83:1467-1473; Salmons and Gunzberg, Human Gene Therapy (1993); 4:129-141; and Grossman and Wilson, Curr. Opin. in Genetics and Devel. (1993); 3:110-114.

Adenoviruses are especially attractive vehicles for delivering genes. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used. Adeno-associated virus (AAV) may also be used (Walsh et al., Proc. Soc. Exp. Biol. Med. 204:289-300 (1993); U.S. Pat. No. 5,436,146).

Another approach to introducing the therapeutic compound to a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

The nucleic acid molecule can be introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny. The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced encompass any desired, available cell type, and include but are not limited to epithelial cells, endothelial cells, keratinocytes, fibroblasts, muscle cells, hepatocytes; blood cells such as Tlymphocytes, Blymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. Recombinant cells can also be used, where nucleic acid sequences encoding an NOX2 or Rubicon or functional fragment thereof, are introduced into the cells such that they are expressible by the cells or their progeny, and the recombinant cells are then administered in vivo for therapeutic effect. For example, stem or progenitor cells can be used. Any stem and/or progenitor cells which can be isolated and maintained in vitro can potentially be used (see e.g. PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

The LAP-related compounds and pharmaceutical compositions disclosed herein are preferably tested in vitro, and then in vivo for the desired therapeutic activity (LAP-related therapeutic activity), prior to use in humans. For example, in vitro assays to demonstrate the therapeutic utility of a compound or pharmaceutical composition include, the effect of a compound on inflammation in a patient tissue sample. The effect of the compound or composition on inflammation of the tissue sample can be determined utilizing techniques known to those of skill in the art.

The terms “vector” and “expression vector” refer to the vehicle by which a DNA or RNA sequence (e.g., a foreign gene) can be introduced into a host cell, so as to transform the host and promote expression (e.g., transcription and translation) of the introduced sequence. Vectors include plasmids, phages, viruses, etc. A “therapeutic vector” as used herein refers to a vector which is acceptable for administration to an animal, and particularly to a human.

Vectors disclosed herein can comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector can comprise coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET plasmids (Invitrogen, San Diego, Calif.), pcDNA3 plasmids (Invitrogen), pREP plasmids (Invitrogen), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g., antibiotic resistance, and one or more expression cassettes.

Suitable vectors include viruses, such as adenoviruses, adeno-associated virus (AAV), vaccinia, herpesviruses, baculoviruses and retroviruses, parvovirus, lentivirus, bacteriophages, cosmids, plasmids, fungal vectors, naked DNA, DNA lipid complexes, and other recombination vehicles typically used in the art which have been described for expression in a variety of eukaryotic and prokaryotic hosts.

Viral vectors, especially adenoviral vectors can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, incorporated herein by reference). AAV vectors, such as those disclosed in U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see McConnell et al., 2004, Hum Gene Ther. 15(11):1022-33; Mccarty et al., 2004, Arum Rev Genet. 38:819-45; Mah et al., 2002, Clin. Pharmacokinet. 41(12):901-11; Scott et al., 2002, Neuromuscul. Disord. 12(Suppl 1):523-9. In addition, see U.S. Pat. No. 5,670,488. Beck et al., 2004, Curr Gene Ther. 4(4): 457-67, specifically describe gene therapy in cardiovascular cells.

### 5. Methods of Identifying Compounds for Modulation of LAP Activity

The methods and compositions disclosed herein include methods for identifying a molecule or composition that modulates LAP activity. Modulating LAP activity refers to increasing or decreasing LAP activity or LAP-related inflammation. LAP activity can be measured by any means known in the art. See, Martinez et al. (2015) Nature Cell Biology 17: 893-906, herein incorporated by reference in the entirety. Specifically, flow cytometry, western blotting (for detecting Rubicon or LC3-II) or immunofluorescence can be used to measure LAP activity. For example, immunofluorescence can be used to identify LC3 association with phagosomes. In some embodiments, LAP activity can be determined by measuring inflammation. For example, measuring inflammation can comprise measuring the level of a pro-inflammatory cytokine, an anti-inflammatory cytokine, or a combination of pro-inflammatory cytokines and anti-inflammatory cytokines. In specific embodiments, measuring inflammation comprises measuring the level of IL-10.

Generally, molecules or compositions that modulate LAP activity can be identified by any screening assay known in the art. For example, a first level of LAP activity can be measured prior to contact with candidate molecules. A second level of LAP activity can then be measured following contact with the candidate molecules. Molecules can be selected based on the relative first and second level of LAP activity, before and after contact with the candidate molecules. Likewise, the level of LAP activity could be measured in a test cell or tissue and in a control cell or tissue following exposure to the candidate molecule. In such an embodiment, the candidate molecule would be selected if the level of LAP activity is modulated in the test cell or tissue when compared to the control cell or tissue. Similarly, the level of LAP activity could be measured following contacting of the candidate molecule with a LAP-deficient cell or tissue. In such an embodiment, the candidate molecule could be selected if LAP activity was restored in the LAP-deficient cell or tissue when compared to a wild type control.

Accordingly, candidate molecules can be selected that modulate (i.e., increase or decrease) the level of LAP activity. A modulated level of LAP activity can be an increase of LAP activity, for instance an increase of at least 1.2, 1.5, 2, 3, 4, 5, 6, 7, 8, 10, 20, 50 times or more relative to an appropriate control. Alternatively, modulation can be a decrease of the level of LAP activity, for instance a decrease of at least 1.2, 1.5, 2, 3, 4, 5, 6, 7, 8, 10, 20, 50 times or more relative to an appropriate control. In some embodiments, the increase or decrease in LAP activity is a statistically significant increase or decrease as determined by methods known in the art.

The cells or tissue used for identifying modulation of LAP activity could be any cell or tissue in which LAP activity can be measured. In some embodiments the cell or tissue is a bone marrow-derived macrophage or a culture of bone marrow-derived macrophages. For example, the bone marrow-derived macrophages can be from an LAP-deficient animal (i.e., mice). In specific embodiments, bone marrow-derived macrophages are isolated from Rubicon deficient mice.

Molecules and compounds isolated by the methods disclosed herein can be formulated as pharmaceutical compositions for administration according to the methods disclosed herein.

### Embodiments

1. A method for decreasing inflammation in a LC3-associated phagocytosis (LAP)-deficient subject comprising administering an effective amount of a pharmaceutical composition that targets the LAP pathway, wherein said administration of an effective amount of a pharmaceutical composition that targets the LAP pathway decreases inflammation.

2. The method of embodiment 1, wherein said LAP-deficient subject has reduced expression of at least one of: Beclin1, VPS34, UVRAG, ATG5, ATG12, ATG16L, ATG7, ATG3, ATG4, LC3A, LC3B, GATE16, GABARAP, Rubicon, or NOX2, when compared to a subject not deficient in LAP.

3. The method of embodiment 1 or 2, wherein said LAP-deficient subject has reduced expression of Rubicon or NOX2 when compared to a subject not deficient in LAP.

4. The method of any one of embodiments 1-3, wherein said pharmaceutical composition that targets the LAP pathway is a peroxisome proliferator-activated receptor (PPAR) agonist or a liver X receptor (LXR) agonist.

5. The method of embodiment 4, wherein said PPAR agonist is a PPAR-γ (gamma) or a PPAR-β/δ (beta/delta) agonist.

6. The method of embodiment 5, wherein said PPAR-γ agonist is a Rosiglitazone.

7. The method of embodiment 5, wherein said PPAR-β/δ agonist is a GW0742.

8. The method of embodiment 4, wherein said LXR agonist is Tesaglitazar.

9. The method of any one of embodiments 1-8, further comprising detecting failed clearance of dead cells prior to administering an effective amount of said pharmaceutical composition.

10. The method of embodiment 9, wherein said failed clearance of dead cells results from a deficiency in the LAP pathway.

11. The method of any one of embodiments 1-10, wherein IL-10 production is increased following administration of an effective amount of said pharmaceutical composition or wherein IL-6 production and/or MCP-1 production decreases following administration of an effective amount of said pharmaceutical composition.

12. A method for treating systemic lupus erythematosus (SLE) comprising administering an effective amount of a pharmaceutical composition that targets the LAP pathway to a subject diagnosed with SLE, wherein said administration of an effective amount of a pharmaceutical composition that targets the LAP pathway decreases at least one symptom of SLE.

13. The method of embodiment 12, wherein said subject has reduced expression of at least one of: Beclin1, VPS34, UVRAG, ATG5, ATG12, ATG16L, ATG7, ATG3, ATG4, LC3A, LC3B, GATE16, GABARAP, Rubicon, or NOX2, when compared to a subject not deficient in LAP.

14. The method of embodiment 12 or 13, wherein said subject has reduced expression of Rubicon or NOX2 when compared to a subject not deficient in LAP.

15. The method of any one of embodiments 12-14, wherein said pharmaceutical composition that targets the LAP pathway is a peroxisome proliferator-activated receptor (PPAR) agonist or a liver X receptor (LXR) agonist.

16. The method of embodiment 15, wherein said PPAR agonist is Rosiglitazone or GW0742.

17. The method of embodiment 15, wherein said LXR agonist is Tesaglitazar.

18. The method of any one of embodiments 12-17, further comprising detecting failed clearance of dead cells prior to administering an effective amount of said pharmaceutical composition.

19. The method of embodiment 18, wherein said failed clearance of dead cells results from a deficiency in the LAP pathway.

20. A method for clearing dead cells in a subject deficient in dead cell-clearance comprising administering an effective amount of a pharmaceutical composition that targets the LAP pathway, wherein said administration of an effective amount of a pharmaceutical composition that targets the LAP pathway decreases inflammation.

21. The method of embodiment 20, wherein said subject is a LAP-deficient subject.

22. The method of embodiment 21, wherein said subject has reduced expression of at least one of: Beclin1, VPS34, UVRAG, ATG5, ATG12, ATG16L, ATG7, ATG3, ATG4, LC3A, LC3B, GATE16, GABARAP, Rubicon, or NOX2, when compared to a subject not deficient in LAP.

23. The method of embodiment 21 or 22, wherein said subject has reduced expression of Rubicon or NOX2 when compared to a subject not deficient in LAP.

24. The method of any one of embodiments 20-23, wherein said pharmaceutical composition that targets the LAP pathway is a peroxisome proliferator-activated receptor (PPAR) agonist or a liver X receptor (LXR) agonist.

25. The method of embodiment 24, wherein said PPAR agonist is Rosiglitazone or GW0742.

26. The method of embodiment 24, wherein said LXR agonist is Tesaglitazar.

27. The method of any one of embodiments 1-26, wherein said administration of an effective amount of the pharmaceutical composition comprises introducing into the subject an expression cassette comprising a LAP nucleic acid sequence encoding a composition that targets the LAP pathway, wherein an effective amount of said pharmaceutical composition is expressed from the expression cassette.

28. The method of embodiment 27, wherein said LAP nucleic acid sequence is operably linked to a promoter active in the subject.

29. The method of embodiments 27 or 28, wherein said LAP nucleic acid sequence encodes Beclin1, VPS34, UVRAG, ATG5, ATG12, ATG16L, ATG7, ATG3, ATG4, LC3A, LC3B, GATE16, GABARAP, Rubicon, or NOX2.

30. The method of any one of embodiments 27-29, wherein said LAP nucleic acid sequence encodes Rubicon or NOX2.

31. The method of any one of embodiments 27-30, wherein said expression cassette is located on a vector.

32. The method of any one of embodiments 27-31, wherein said expression cassette is stably incorporated in the genome of said subject following said introducing step.

33. The method of any one of embodiments 27-32, wherein introducing the expression cassette comprises introducing a cell comprising said expression cassette.

34. The method of embodiment 33, wherein said expression cassette is located on a vector.

35. A method of identifying a molecule that modulates LAP activity comprising:
measuring a first level of LAP activity in a cell or tissue;
contacting the cell or tissue with a candidate compound;
measuring a second level of LAP activity of said cell or tissue after said contacting with a candidate compound;
comparing said first level of LAP activity with the second level of LAP activity; and
selecting compounds that modulate the LAP activity.

36. A method of identifying a molecule that modulates LAP activity comprising:
contacting a test cell or tissue with a candidate compound;
measuring a first level of LAP activity of said test cell or tissue after said contacting with a candidate compound;
measuring a second level of LAP activity from a control cell or tissue;
comparing said first level of LAP activity with said second level of LAP activity; and
selecting compounds that modulate the LAP activity.

37. The method of embodiment 35 or 36, wherein compounds are selected that increase or decrease LAP activity.

38. The method of any one of embodiments 35-37, wherein measuring said first and second level of LAP activity comprises measuring inflammation.

39. The method of embodiment 38, wherein measuring inflammation comprises measuring the level of at least one pro-inflammatory or at least one anti-inflammatory cytokine, or a combination of pro-inflammatory and anti-inflammatory cytokines.

40. The method of embodiment 38 or 39, wherein measuring inflammation comprises measuring the level of IL-10, IL-6, and/or MCP-1.

41. The method of any one of embodiments 35-40, wherein said cell or tissue is a bone marrow-derived macrophage or a culture of bone marrow-derived macrophages.

42. The method of embodiment 41, wherein said bone marrow-derived macrophage is generated from LAP-deficient mice.

43. The method of embodiment 42, wherein said LAP-deficient mice are Rubicon deficient.

44. The method of any one of embodiments 35-43, wherein said selected molecule modulates LAP activity when administered to a subject.

45. The method of embodiment 44, wherein said subject has an inflammatory disease.

46. A pharmaceutical composition comprising a molecule selected by the method of any one of embodiments 35-45.

47. Use of a pharmaceutical composition that targets the LAP pathway for decreasing inflammation or treating SLE according to the methods of embodiments 1-11 or 12-19, respectively.

48. Use of a pharmaceutical composition that targets the LAP pathway according to the method of any one of embodiments 1-45.

49. A pharmaceutical composition that targets the LAP pathway for use in treating an inflammatory disorder or SLE in a LAP-deficient subject, said use comprising administering an effective amount of a pharmaceutical composition that targets the LAP pathway to the subject.

50. The pharmaceutical composition of embodiment 49, wherein said subject has reduced expression of at least one of: Beclin1, VPS34, UVRAG, ATG5, ATG12, ATG16L, ATG7, ATG3, ATG4, LC3A, LC3B, GATE16, GABARAP, Rubicon, or NOX2, when compared to a subject not deficient in LAP.

51. The pharmaceutical composition of embodiments 49 and 50, wherein said pharmaceutical composition that targets the LAP pathway is a peroxisome proliferator-activated receptor (PPAR) agonist or a liver X receptor (LXR) agonist.

52. The pharmaceutical composition of any one of embodiments 49-51, wherein the pharmaceutical composition increases LAP activity.

### EXPERIMENTAL

### Example 1. Treatment of LAP-Deficient Mice with PPAR and LXR Agonists to Restore IL-10 Production

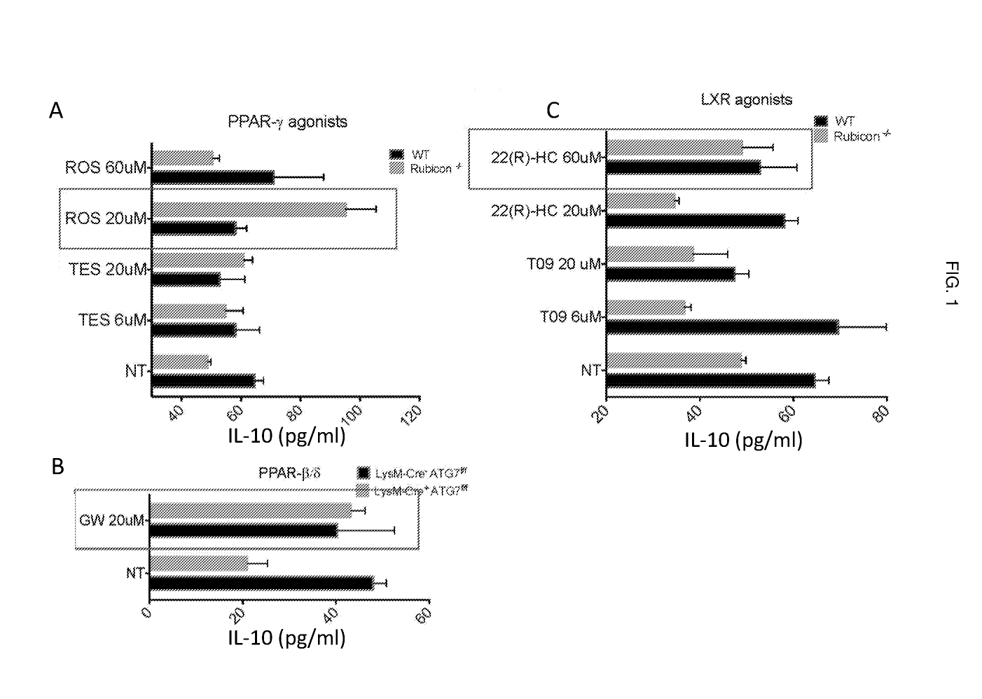

Bone marrow-derived macrophages (BMM) were generated as previously described from Rubicon−/− mice and wild-type littermates. Macrophages were co-cultured with UV-irradiated apoptotic thymocytes (at a ratio of 10 apoptotic cells:1 macrophage) in the presence or absence of PPARγ agonists Rosiglitazone (ROS, 20 or 60 μM) or Tesaglitazar (TES, 6 or 20 μM). NT indicates no treatment. After 18 hours of co-culture, supernatants were collected and analyzed for IL-10 production via ELISA (FIG. 1A). Administration of 20 μM of Rosiglitazone increased IL-10 production in Rubicon-deficient (LAP-deficient) mice, beyond that of the control treatment. Thus, PPAR agonists can be effective at restoring the LAP phenotype in LAP-deficient cells.

Bone marrow-derived macrophages (BMM) were generated as previously described from LysM-Cre− ATG7f/f and LysM-Cre+ ATG7f/f mice. Macrophages were co-cultured with UV-irradiated apoptotic thymocytes (at a ratio of 10 apoptotic cells:1 macrophage) in the presence or absence of PPARβ/δ agonist GW0742 (GW, 20 μM). NT indicates no treatment. After 18 hours of co-culture, supernatants were collected and analyzed for IL-10 production via ELISA (FIG. 1B). Administration of 20 μM of GW0742 increased IL-10 production in LysM⁻Cre⁺ ATG7^{f/f} (LAP-deficient) mice, beyond that of the control treatment. Thus, PPAR agonists can be effective at restoring the LAP phenotype in LAP-deficient cells.

Bone marrow-derived macrophages (BMM) were generated as previously described from Rubicon−/− mice and wild-type littermates. Macrophages were co-cultured with UV-irradiated apoptotic thymocytes (at a ratio of 10 apoptotic cells:1 macrophage) in the presence or absence of LXR agonists T0901317 (T09, 6 or 20 μM) or 22(R)-hydroxycholesterol (22(R)-HC, 20 or 6 μM). NT indicates no treatment. After 18 hours of co-culture, supernatants were collected and analyzed for IL-10 production via ELISA FIG. 1C). Administration of 60 μM of 22(R)-hydroxycholesterol increased IL-10 production in Rubicon-deficient (LAP-deficient) mice, beyond that of the control treatment. Thus, LXR agonists can be effective at restoring the LAP phenotype in LAP-deficient cells.

### Example 2. Noncanonical Autophagy Inhibits the Auto-Inflammatory, Lupus-Like Response to Dying Cells

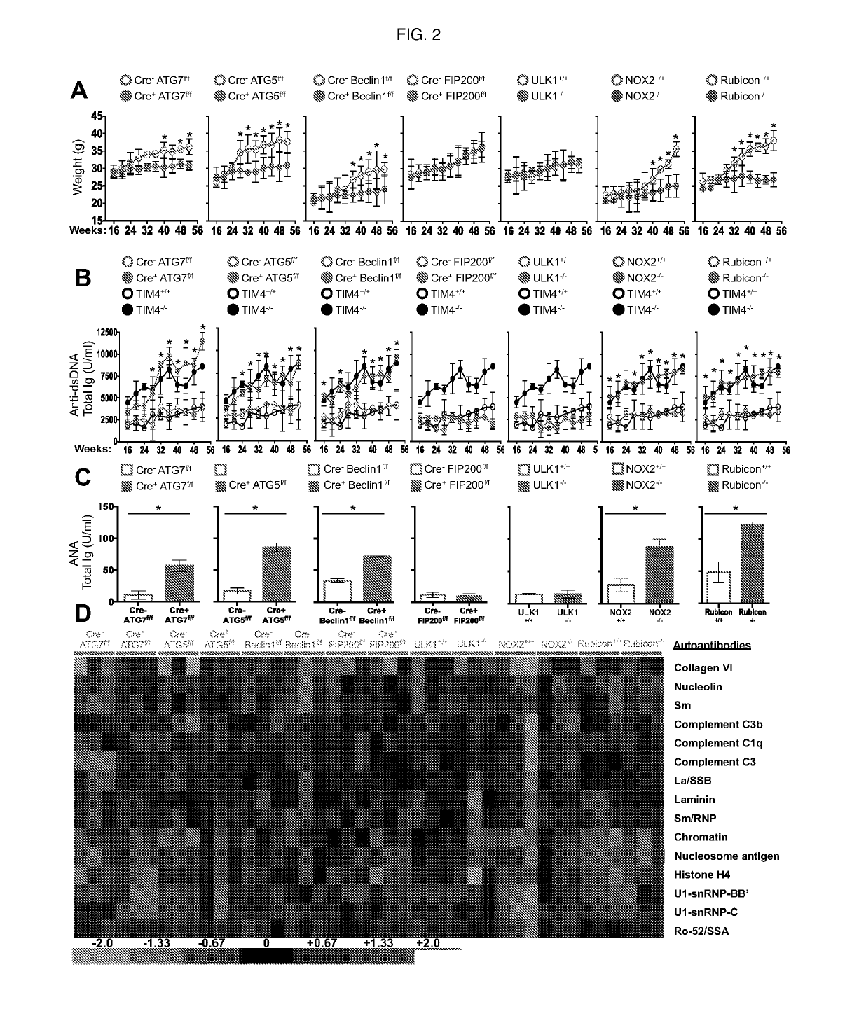

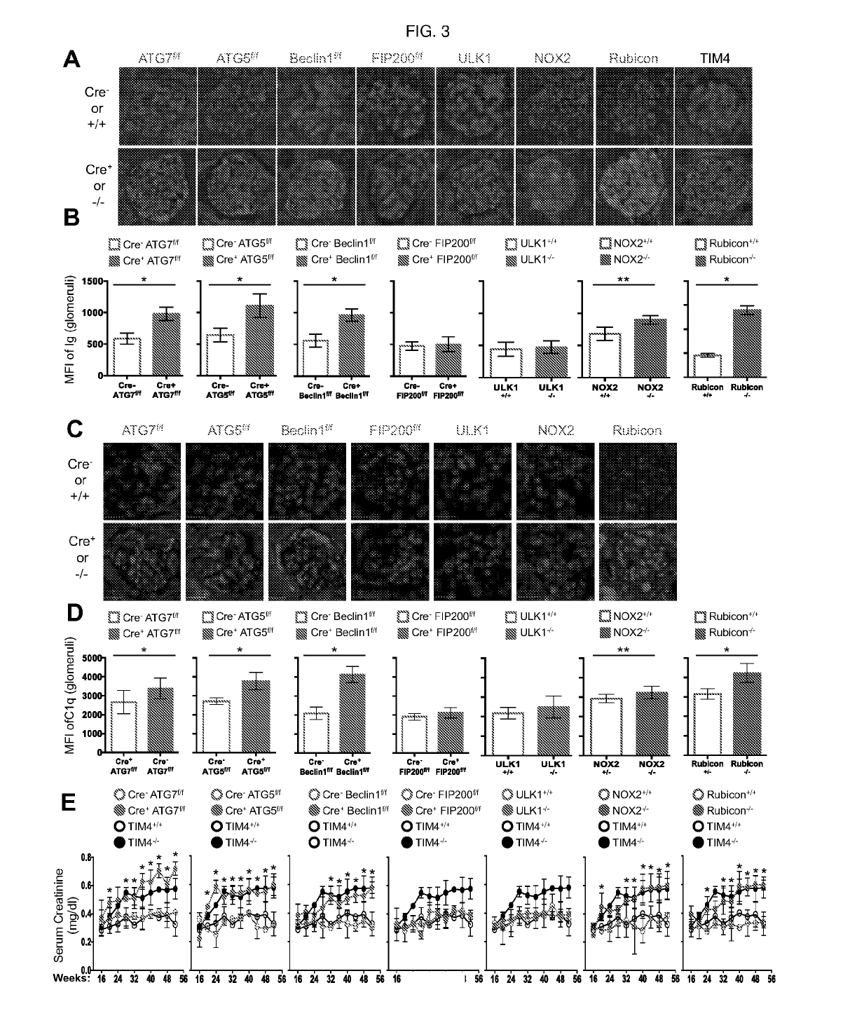

As many components of autophagy are required for development (e.g., FIP200^{11,12}, Beclin 1¹²) or post-natal survival (e.g., ATG14^{12,13}, ATG7¹², ATG5¹², ATG16L¹²), animals were generated in which several autophagy genes were conditionally ablated using LysM-Cre¹⁴, affecting macrophages (CD11b⁺/F4/80⁺), monocytes (CD11b⁺/CD115⁺), some neutrophils (CD11b⁺/Ly6G⁺), and some conventional dendritic cells (CD11b⁺/CD11c⁺), but not eosinophils, plasmacytoid dendritic cells, or lymphocytes. While all animals appeared normal at weaning, we observed that LAP-deficient genotypes failed to gain weight compared to their wild-type (WT) littermates (FIG. 2A). This effect was observed in animals lacking proteins required for both LAP and autophagy (ATG7, ATG5, Beclin 1) or LAP alone (NOX2, Rubicon), but not in animals lacking proteins required for autophagy but dispensable for LAP (FIP200, ULK1). Compared to LAP-sufficient animals, LAP-deficient mice displayed elevated circulating lymphocytes, monocytes, and neutrophils, with elevated circulating activated CD8⁺ T cells, and increased immunohistological staining of CD3 and Ki67 in the spleen. Strikingly, LAP-deficient animals also contained increased serum levels of anti-dsDNA antibodies and anti-nuclear antibodies (FIG. 2B-C), as well as a broad array of antibodies against autoantigens commonly associated with SLE (FIG. 2D). LAP-deficient animals also presented with IgG and complement C1q deposition in glomeruli of kidneys (FIG. 3A-D). In addition, LAP-deficient animals displayed indications of kidney damage¹⁵, and exhibited increased functional markers of kidney injury, such as elevated serum creatinine (FIG. 3E), blood urea nitrogen (BUN), and proteinuria (ACR). Histologically, kidneys from aged LAP-deficient animals displayed endocapillary proliferative glomerulonephriti. Increased expression of type I interferon (IFN) regulated genes, termed the IFN signature, has been reported in SLE patients¹⁶. Analysis revealed increased expression of IFN signature genes, such as Ddx58 (which encodes RIG-I) and Isg95, in the spleens of aged LAP-deficient animals. In contrast, none of these pathologies were observed in animals lacking autophagy components dispensable for LAP (FIG. 3A-E). Collectively, these observations suggest that LAP deficiency, but not autophagy deficiency, causes an autoinflammatory, lupus-like syndrome in mice.

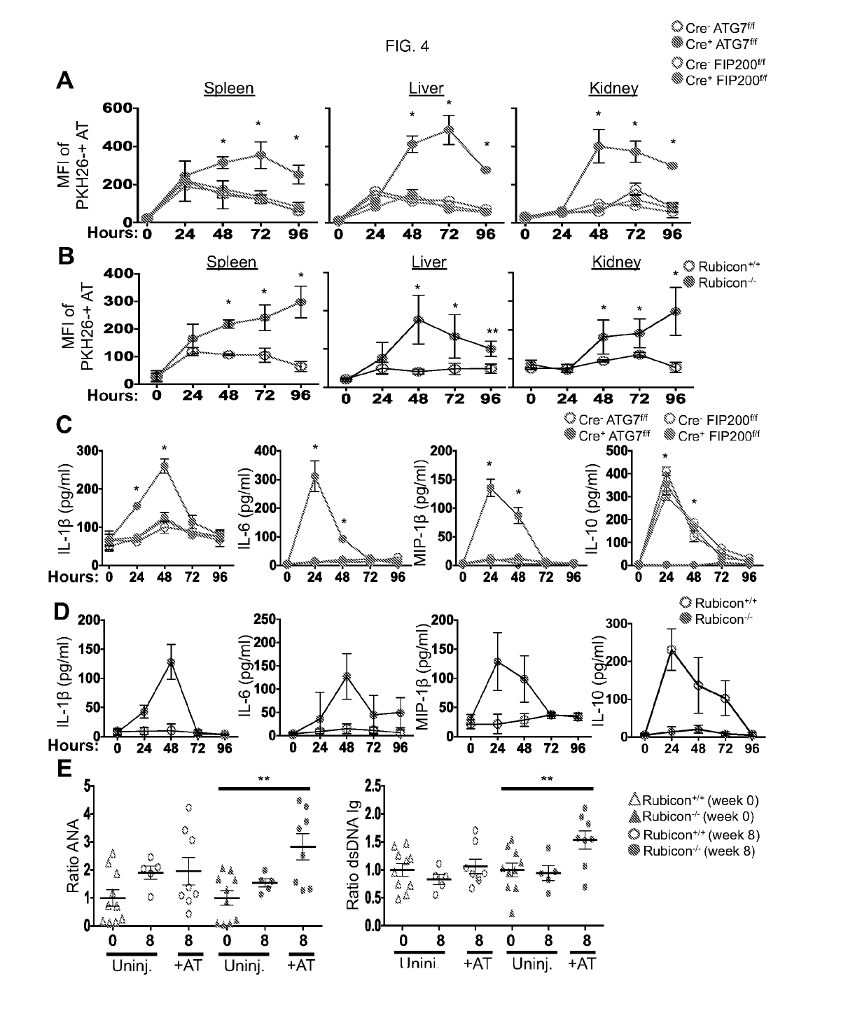

The kinetics of disease we observed in all LAP-deficient animals was strikingly similar to that of animals lacking T-cell immunoglobulin mucin protein 4 (TIM4) (FIG. 2A-B, 3A, 3E). TIM4 is required for engulfment of dying cells in several macrophage populations, and animals lacking TIM4 display lupus-like disease², as do animals defective for other proteins involved in the clearance of dying cells, including Mertk, MFG-E8, and C1q¹. However, neither bone marrow-derived macrophages, nor peritoneal exudate macrophages from 52-week old mice of any genotype showed any defects in the engulfment of dying cells in vitro. The role of LAP in the response to dying cells in vivo was examined. PKH26-labelled WT C57Bl/6 thymocytes were UV-irradiated to trigger apoptosis and immediately injected into WT animals, or animals with LysM-Cre-mediated deficiency of ATG7 (LAP-deficient, autophagy-deficient), LysM-Cre-mediated deficiency of FIP200 (LAP-sufficient, autophagy-deficient), or ubiquitous deletion of Rubicon (LAP-deficient, autophagy-sufficient), all of which also expressed transgenic GFP-LC3⁵. Clearance of dying thymocytes and induction of LC3-II (a measure of LC3 conversion⁵) were monitored in spleen, liver, and kidney. While both WT and animals with FIP200-deficiency effectively cleared dying cells (FIG. 4A-B) and converted GFP-LC3, animals with ATG7- or Rubicon-deficiency did not, despite engulfment (FIG. 4A-B). These data are consistent with previous observations in vitro⁴ and support the conclusion that LAP is required for effective degradation of engulfed, dying cells in vivo. Dying cells were engulfed by CD11b⁺/F4/80⁺ macrophages, CD11b⁺/Gr1⁺ granulocytes, CD11b⁺/CD115⁺ monocytes, and CD11b⁺/CD11c⁺ dendritic cells, equivalently in WT and Rubicon^{-/-} mice, but not in TIM4^{-/-} mice. However, while frequency of engulfment declined by 48 hours in all cellular subsets in WT mice, they remained elevated in Rubicon^{-/-} mice, consistent with a failure of a LAP-dependent mechanism to degrade engulfed corpses.

In contrast to WT or ULK1^{-/-} macrophages, ATG7^{-/-} macrophages produce increased levels of inflammatory cytokines, such as IL-1β and IL-6 in vitro⁴. We therefore examined cytokine production upon ingestion of dying cells in macrophages lacking different components of the LAP or autophagy pathways. LAP-deficient (Cre⁺ ATG7^{flox/flox}, Cre⁺ Beclin 1^{flox/flox}, Cre⁺ ATG3^{flox/flox} NOX2^{-/-}, and Rubicon^{-/-}) but not LAP-sufficient (Cre⁺ FIP200^{flox/flox}, Cre⁺ ATG14^{flox/flox}) macrophages produced IL-1β, IL-6, and IP-10/CXCL10, upon engulfment of dying cells. Conversely, LAP-sufficient, but not LAP-deficient macrophages produced IL-10 upon engulfment. The effects of dying cells on serum cytokine production in vivo, following injection of UV-irradiated thymocytes was also examined (FIG. 4C-D). Strikingly, serum IL-1β, IL-6, and MIP-1β/CCL4 were acutely elevated in LAP-deficient animals (ATG7 or Rubicon), but not in LAP-sufficient animals (WT or FIP200) (FIG. 4C-D). As we had observed in vitro, LAP-sufficient animals produced elevated serum IL-10 in response to dying cells, while LAP-deficient animals did not (FIG. 4C-D). Therefore, LAP, but not canonical autophagy, is required for the production of IL-10 in response to apoptotic cell engulfment, and LAP suppresses production of inflammatory cytokines under these conditions.

Repeated injection of apoptotic thymocytes into LAP-deficient animals was examined to determine if such repeated injection could exacerbate the SLE-like phenotype observed in aged LAP-deficient animals. Beginning at 6 weeks of age, Rubicon^{+/+} and Rubicon^{-/-} animals were injected with UV-irradiated thymocytes over an 8-week period. Uninjected Rubicon^{+/+} animals showed a minimal increase in ANA and anti-dsDNA autoantibodies after 8 weeks, and no increase attributable to injection of dying cells. Rubicon^{-/-} animals, however, displayed a significant increase in serum levels of ANA and anti-dsDNA autoantibodies after 8 weeks of dying cell injections, above pre-injection and age-matched, uninjected controls (FIG. 4E). Further, these animals displayed IgG and C1q deposition in glomeruli of kidneys, and injected Rubicon^{-/-} animals displayed elevated levels of alanine aminotransferase (ALT), indicative of tissue damage. Collectively, these data demonstrate that defective dead cell clearance associated with LAP deficiency can result in development of SLE-like disease.

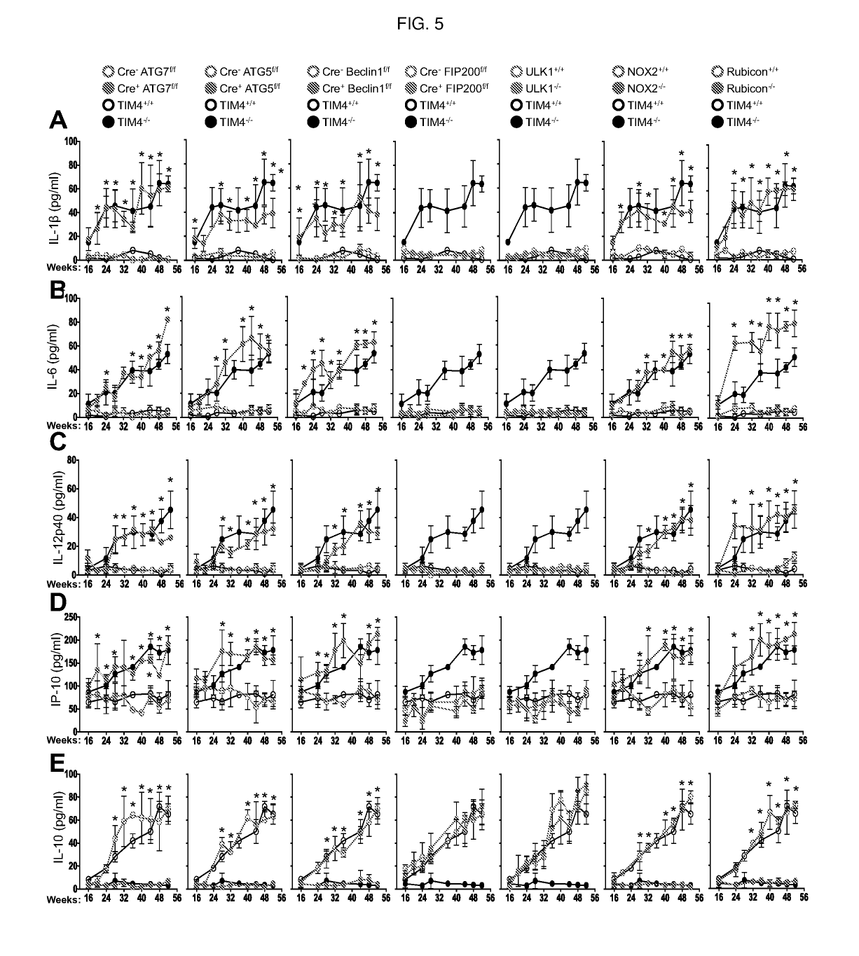

The spontaneous levels of serum cytokines with age in animals with or without LAP was examined. All genotypes lacking LAP (Cre⁺ ATG7^{flox/flox}, Cre⁺ ATG5^{flox/flox}, Cre⁺ Beclin 1^{flox/flox}, NOX2^{-/-}, and Rubicon^{-/-}) displayed elevated IL-1β, IL-6, IL-12p40, and IP-10/CXCL10, (FIG. 5A-D) as well as KC/CXCL1, MIP-1β/CCL4, and MCP-1/CCL2. Wild-type animals and animals lacking canonical autophagy, but not LAP (in monocytes or systemically), did not display elevated inflammatory cytokines at any time point (FIG. 5A-D). In contrast, serum IL-10 levels, which increased with age in LAP-sufficient strains, were undetectable in animals lacking LAP (FIG. 5E). The patterns and kinetics of cytokine levels were similar to that observed in TIM4^{-/-} animals (FIG. 5A-E).

These observations indicated that defects in LAP, but not canonical autophagy, cause an autoinflammatory, lupus-like syndrome in mice. To further test this idea, both LAP-sufficient and LAP-deficient mice bred in an independent facility were tested. Mice with ATG5- or ATG3-deficient myeloid cells (defective in LAP and autophagy) displayed increased levels of elevated IL-1β, IL-6, IL-12p40, IP-1/CXCL10, KC/CXCL1, MIP-1β/CCL4, and MCP-1/CCl2 at 52-weeks of age. These LAP-deficient animals also displayed significantly lower levels of IL-10, compared to controls. Furthermore, LAP-deficient animals displayed elevated anti-dsDNA antibodies and serum creatinine. LAP-deficient animals also contained a broad array of antibodies against autoantigens commonly associated with SLE. Of note, none of these effects were observed in animals with ATG14- or FIP200-deficiency (defective autophagy but normal LAP^{3,6,7,11,13}). It is noteworthy that these effects in two different facilities were observed in C57Bl/6 background animals, which is generally resistant to lupus-like disease¹⁷.

Altogether, these data suggest that defective LAP results in a failure to digest engulfed dying cells, leading to elevated inflammatory cytokine production and a lupus-like syndrome. In another study, animals in which lung macrophages were incapable of engulfment due to deletion of Rac 1 were sensitive to inflammatory cytokine production and inflammatory disease upon introduction of dying cells into the lung¹⁹. Similarly, TIM4-deficient mice, which exhibit defective dead cell engulfment², showed spontaneous elevation of serum inflammatory cytokines with age (FIG. 5) as well as lupus-like disease (FIG. 2, 3). In contrast, macrophages defective for LAP engulf dying cells, but fail to efficiently digest them^{4,7}. This suggests that LAP-dependent digestion of dying cells, rather than engulfment alone, suppresses an inflammatory response by macrophages. In the absence of LAP (lack of Beclin 1, ATG7, ATG5, NOX2, Rubicon), macrophages engulf dying cells and produce inflammatory cytokines, and animals manifest lupus-like disease. However, when canonical autophagy, but not LAP, is defective (lack of FIP200, ULK1), dying cells are engulfed, macrophages produce IL-10 but not inflammatory cytokines, and no lupus-like disease is observed.

MRL.lpr mice lacking IL-10 display dramatically accelerated lupus-like disease²⁰. While macrophages, monocytes, and B cells are the major source of IL-10, specific deletion of IL-10 in B cells had no effect on pathogenesis in MRL.lpr mice²¹. Intriguingly, one study found that injection of dendritic cells that had engulfed necrotic cells into IL-10-deficient, but not WT mice induced a pronounced lupus-like disease²². Thus, the role of LAP in the production of IL-10 may contribute to the disease effects we observed. However, most studies have implicated elevated IL-10 levels in mouse and human SLE^{20,23,24}, perhaps involved with activation of B lymphocytes²⁵. While IL-10 production in response to dying cells was compromised in LAP-deficient macrophages, the production of IL-10 in response to other stimuli may remain intact, and thus elevated IL-10 in SLE may be due to other events in the pathogenesis of SLE.

Genome-wide association studies have implicated autophagy in SLE (Atg5^{6,8}, and possibly Atg7⁹) and in Crohn's disease (Atg16l²⁶). It is noteworthy in this context that the ATG5 association with SLE may depend on polymorphisms in IL-10^{8,27}. Other studies have suggested that autophagy suppresses the inflammasome²⁸, providing a possible link between autophagy and inflammatory disease. However, the autophagic components identified in these studies are also required for LAP. Further, mice¹⁸ and humans²⁹, lacking NOX2 develop SLE, and these studies suggest that defective LAP in this context may contribute to this effect. Our findings implicate a noncanonical autophagic process, LAP, in the control of inflammatory disease and suggest a link between the clearance of dying cells, autophagic processes, and inflammation in the control of SLE.

All mice were housed specific pathogen-free. ULK1^{-/-} mice were kindly provided by Mondira Kundu (St. Jude Children's Research Hospital). ATG7^{flox/flox} mice (kindly provided by Masaaki Komatsu at The Tokyo Metropolitan Institute of Medical Science) were bred to LysM-Cre⁺ mice (kindly provided by Peter Murray, St. Jude Children's Research Hospital) and GFP-LC3⁺ mice to generate LysM-Cre⁺ ATG7^{flox/flox} GFP-LC3⁺ versions of these strains. NOX2^{-/-} mice were purchased from Jackson Laboratories. LysM-Cre⁺ Beclin 1^{flox/flox} (Edmund Rucker, University of Kentucky), LysM-Cre⁺ ATG5^{flox/flox} (Thomas A. Ferguson, Washington University), and LysM-Cre⁺ FIP200^{flox/flox} (Jun-Lin Guan, University of Michigan) were bred to GFP-LC3⁺ mice to generate GFP-LC3⁺ versions of these strains. TIM4^{-/-} mice were kindly provided by Vijay Kuchroo (Harvard University). Rubicon^{+/+} and Rubicon^{-/-} mice were generated using CRISPR/Cas9 gene editing technology⁵. LysM-Cre⁺ ATG14^{flox/flox}, LysM-Cre⁺ ATG3^{flox/flox}, LysM-Cre⁺ ATG5^{flox/flox}, LysM-Cre⁺ FIP200^{flox/flox} mice (and control littermates) were bred and maintained in the Washington University (WU) facility. The St. Jude Institutional Animal Care and Use Committee approved all procedures in accordance with the Guide for the Care and Use of Animals.

Bone marrow-derived macrophages (BMDMs) were generated from bone marrow progenitors obtained from littermates. Freshly prepared bone marrow cells were cultured in DMEM medium supplemented with 10% heat-inactivated FCS, 2 mM L-glutamine, 10 mM HEPES buffer, 50 μg/ml penicillin, and non-essential amino acids in the presence of 20 ng/ml rmM-CSF (Peprotech) for 6 days. Nonadherent cells were removed on day 6, and adherent macrophages were detached from plates and re-plated for experimental use.

Male wild-type and knockout littermates were co-housed and allowed to age for 52 weeks. Animals were weighed and bled retro-orbitally monthly, and serum was collected for use in assays (below). Numbers of animals were as follows (in all cases, Cre indicates LysM-Cre.) Studies conducted at St. Jude Children's Research Hospital and reported in FIGS. 1, 2, 4, D2, S3, S4, S5, and S8: Cre⁻ and Cre⁺ ATG7^{f/f}, n=24 per genotype; Cre⁻ and Cre⁺ ATG5^{f/f}, n=14 per genotype; Cre⁻ and Cre⁺ Beclin1^{f/f}, n=20 per genotype; Cre⁻ and Cre⁺ FIP200^{f/f}, n=16 per genotype; ULK1^{+/+} and ULK1^{-/-}, n=14 per genotype; NOX2^{+/+} and NOX2^{-/-}, n=10 per genotype; Rubicon^{+/+} and Rubicon^{-/-}, n=14 per genotype. Studies conducted at Washington University and reported in FIGS. S9 and S10: Cre⁻ and Cre⁺ ATG5^{f/f}, n=5 per genotype; Cre⁻ and Cre⁺ ATG3^{f/f}, n=4 per genotype; Cre⁻ and Cre⁺ FIP200^{f/f}, n=4 per genotype; Cre⁻ and Cre⁺ ATG14^{f/f}, n=4 per genotype.

Apoptosis was induced in wild-type C57Bl/6 thymocytes by UV irradiation (20 J/m²). Thymocytes were washed twice with PBS prior to experimental use.

UV-treated thymocytes were stained with 20 M PKH26 Red (Sigma), per manufacturer's instructions. 1×10⁷ PKH26-labelled, apoptotic thymocytes were injected intravenously into GFP-LC3+ animals, and serum, kidney, liver, and spleen was collected at 0, 24, 48, 72, and 96 hours post-injection. Kidney sections were analyzed for persistence of PKH26-labelled apoptotic cells using the Nikon800 microscope. Kidney, liver, and spleen samples were analyzed for PKH26-labelled apoptotic cells using flow cytometry. Additionally, samples were washed once with FACS buffer and permeabilized with digitonin (Sigma, 200 μg/ml) for 15 minutes on ice. Cells were then washed 3 times with FACS buffer and analyzed by flow cytometry for membrane-bound GFP-LC3-II associated with engulfed PKH26-labeled thymocytes. For quantification of phagocytosis, spleens were harvested and stained for fluorescently conjugated surface markers for macrophages (CD11b⁺ F4/80⁺), neutrophils (CD11b⁺ Gr-1⁺), monocytes (CD11b⁺ CD115⁺), and dendritic cells (CD11b⁺ CD11c⁺). Phagocytic efficiency of each cell type (Singlets/cell surface markers⁺/PKH26⁺) was quantified by flow cytometry (% PKH26).

Six-week-old Rubicon^{+/+} and Rubicon^{-/-} littermates were used. Serum was collected from all animals prior to injection (week 0). 2.0×10⁷ UV-irradiated thymocytes (20 J/m²) suspended in sterile phosphate buffer were injected i.v. into anesthesized mice, once a week for 4 consecutive weeks (from weeks 1 to 4). After a resting period of 15 days, the injections were resumed and carried out for other 2 weeks (weeks 6 and 7). Serum was collected one week after the last injection (week 8) and assessed for levels of anti-dsDNA autoantibodies (Total Ig), anti-nuclear autoantibodies (ANA, Total Ig), and alanine aminotransferase (ALT). At week 8, mice were euthanized, the kidneys were harvested, and stained for immunofluorescence (below).

For peritoneal exudate cell harvests, mice were injected i.p. with 2 ml of 3% Brewer's thioglycollate and euthanized 96 h later. The peritoneum was washed with 10 ml ice cold PBS three times. Cells were centrifuged (1,000×RPM, 6 minutes, 4° C.) and washed twice with sterile PBS. Peritoneal exudate cells were resuspended in DMEM/10% FBS, counted, and plated at 5×10⁵ cells/well in a 12-well plate. Cells were allowed to settle for 2 h (37° C./5% CO2) before co-culture with UV-irradiated wild-type thymocytes.

Apoptotic thymocytes were added to BMDM cultures at a ratio of 10:1 (dead cell:macrophage). Supernatant was collected after 24 hours of culture and analyzed for cytokines (see below).

Spleens, livers, and kidneys were harvested from animals at the indicated time-points, and single cell suspensions were generated. Cells were washed once with FACS buffer, and permeabilized with digitonin (Sigma, 200 μg/ml) for 15 minutes on ice. Cells were then washed 3 times with FACS buffer and analyzed by flow cytometry for membrane-bound GFP-LC3-II. This assay removes the soluble, cytosolic form of GFP-LC3 (GFP-LC3-I), while the lipidated, membrane-bound GFP-LC3-II is retained, allowing total GFP fluorescence to be used as a measure of LC3-II generation, indicative of LAP. Permeabilized samples were first gated on Singlets/PKH26⁺, so as to determine the mean fluorescence intensity (MFI) of GFP-LC3-II associated with cells that had engulfed a PKH26⁺ apoptotic thymocyte. For surface staining, blood, bone marrow, or splenoyctes were washed once with FACS buffer, incubated with Fc Block and stained with the indicated fluorescent antibodies (Biolegend) on ice for 20 minutes. Cells were then washed twice with FACS buffer and analyzed by flow cytometry. Data were acquired using an LSRII cytometer (BD).

Phagocytosis was quantified using flow cytometry analysis (described above). Apoptotic thymocytes were stained with CellTrace Violet (Molecular Probes) or PKH26 (Sigma-Aldrich) per manufacturer's protocol. Percent phagocytosis equals the percentage of cells that have engulfed CellTrace Violet⁺ or PKH26⁺ apoptotic thymocytes.

Kidneys were harvested from animals at 32 weeks, 52 weeks, or 8 weeks after chronic apoptotic thymocyte injection (above). Organs were sectioned and mounted on slides. Slides were fixed with 4% formaldehyde for 20 minutes at 4° C. Following fixation, slides were blocked and permeabilized in block buffer (1% BSA, 0.1% Triton in PBS) for 1 hour at RT. Slides were washed extensively in TBS-Tween (Tris-buffered saline containing 0.05% Tween-20), incubated with Alexa-Fluor 647-conjugated anti-IgG (Invitrogen) for 1 hour at RT, and mounted with VectaShield with DAPI (Vector Labs). Alternatively, slides were washed extensively in TBS-Tween (Tris-buffered saline containing 0.05% Tween-20), incubated with anti-C1q (clone 4.8, Abcam) for 1 hour at RT, washed again with TBS-Tween, incubated with Cy3 conjugated donkey anti-rabbit IgG (Jackson ImmunoResearch) and Alexa Fluor 488-conjugated wheat germ agglutinin (Molecular Probes) for 1 hour at RT, and mounted with VectaShield with DAPI (Vector Labs). Images were analyzed using an Olympus BX51 FL Microscope and Slidebook software. Masks were drawn around glomeruli, and MFI of anti-IgG or anti-C1q were calculated.

Supernatants were collected from macrophages fed with apoptotic thymocytes for 24 hours. Cytokines released into supernatant were analyzed by Luminex technologies (Millipore). Serum was collected from animals was analyzed by Luminex technologies (Millipore).

The Veterinary Pathology Core at St. Jude Children's Research Hospital measured serum creatinine. The Veterinary Pathology Core at St. Jude Children's Research Hospital assessed differential blood counts, alanine aminotransferase (ALT), and proteinuria (albumin to creatitine ratio, ACR). The Clinical Pathology Core at the National Institute of Environmental Health Sciences performed blood urea nitrogen (BUN) analysis.

Kidneys were harvested from 52-week-old mice. Organs were sectioned, fixed in 10% formalin, and embedded in paraffin. Four to six μm serial sections were cut, deparaffinized, rehydrated and stained with hematoxylin and eosin (H&E). All slides were coded prior to evaluation, and only decoded upon collection of all data. Endocapillary proliferative glomerulonephritis (EPG), a glomerular disease pattern frequently associated with lupus nephritis, was assessed on a virtual scale ranging from 0 to 5, where “0” was considered “indistinguishable compared to wild type control” and “5” was considered “the maximal damage seen in all samples”, based on the classification of glomerulonephritis in systemic lupus erythematosus³⁰. Features that influence this score are intraglomerular mesangial proliferation in relation to overall glomerular size, number of mesangial nuclei, intraluminal diameters of glomerular capillaries and the amount of mesangial matrix. Hematoxylin/eosin stained sections were used to score at least 24 glomeruli in a maximum of 4 different specimens obtained from each group.

The presence of anti-dsDNA antibodies in serum was tested using Mouse Anti-dsDNA Ig's (Total A+G+M) ELISA Kit (Alpha Diagnostics International), per manufacturer's protocol. The presence of anti-nuclear antibodies (ANA) in serum was tested using Mouse ANA/ENA Ig's (Total A+G+M) ELISA Kit (Alpha Diagnostics International), per manufacturer's protocol.

Autoantibody reactivities against a penal of 124 autoantigens were measured using an autoantigen microarray platform developed by University of Texas Southwestern Medical (the website at microarray.swmed.edu/products/category/protein-array/). Briefly, serum samples were pretreated with DNAse-I and then diluted 1:50 in PBST buffer for autoantibody profiling. The autoantigen array bearing 124 autoantigens and 4 control proteins were printed in duplicates onto Nitrocellulose film slides (Grace Bio-Labs). The diluted serum samples were incubated with the autoantigen arrays, and autoantibodies were detected with cy3-labeled anti-mouse IgG and cy5-labeled anti-mouse IgM using a Genepix 4200A scanner (Molecular Device) with laser wavelength of 532 nm and 635 nm. The resulting images were analyzed using Genepix Pro 6.0 software (Molecular Devices). The median of the signal intensity for each spot were calculated and subtracted the local background around the spot, and data obtained from duplicate spots were averaged. The background subtracted signal intensity of each antigen was normalized to the average intensity of the total mouse IgG, which was included on the array as an internal control. Finally, the net fluorescence intensity (NFI) for each antigen was calculated by subtracting a PBS control which was included for each experiment as negative control. Signal-to-noise ratio (SNR) was used as a quantitative measurement of the true signal above background noise. SNR values equal to or greater than 3 were considered significantly higher than background, and therefore true signals. The NFI of each autoantibody was used to generate heatmaps using Cluster and Treeview software (rana.bl.gov/EisenSoftware.htm). Each row in the heatmap represents an autoantibdy and each column represents a sample. Red color represents the signal intensity higher than the mean value of the raw and green color means signal intensity is lower than the mean value of the raw.

Total RNA was isolated from the spleens from 52-week-old mice using NucleoSpin II kit (Macherey-Nagel) according to the manufacturer's instructions, and 50 ng was used to determine the absolute levels of gene expression. Hybridization and nCounter were performed according to the manufacturer's protocol (Nanostring Technologies, Seattle, Wash., USA). In brief, reactions were hybridized for 20 h at 65°, after which the products were used to run on the nCounter preparation station for removal of excess probes. Data were collected with the nCounter digital analyzer by counting individual barcodes. Data generated from the nCounter digital analyzer were examined with the nCounter digital analyzer software system v2.1.1 (Nanostring Technologies). Data were normalized to the geometric means of spiked-in positive controls (controls for assay efficiency) and spiked-in negative controls (normalized for background). The data were further normalized to the housekeeping genes Gapdh, Hprt, and Tubb5 and are reported as normalized RNA counts (means±SEM). Nanostring RNA counts were analyzed with the Partek Genomic Suite (Partek, Inc., St. Louis, Mo., USA), to identify significantly regulated probe. Heatmaps of Nanostring data were generated with the Partek Genomic Suite.

The statistical significance of differences in mean values was calculated using unpaired, two-tailed Student's t test. p values less than 0.05 were considered statistically significant.

### REFERENCES

1 Ravichandran, K. S. Find-me and eat-me signals in apoptotic cell clearance: progress and conundrums. The Journal of experimental medicine 207, 1807-1817, doi:10.1084/jem.20101157 (2010).
2 Rodriguez-Manzanet, R. et al. T and B cell hyperactivity and autoimmunity associated with niche-specific defects in apoptotic body clearance in TIM-4-deficient mice. Proceedings of the National Academy of Sciences of the United States of America 107, 8706-8711, doi:10.1073/pnas.0910359107 (2010).
3 Kim, J. Y. et al. Noncanonical autophagy promotes the visual cycle. Cell 154, 365-376, doi:10.1016/j.cell.2013.06.012 (2013).
4 Martinez, J. et al. Microtubule-associated protein 1 light chain 3 alpha (LC3)-associated phagocytosis is required for the efficient clearance of dead cells. Proceedings of the National Academy of Sciences of the United States of America 108, 17396-17401, doi:10.1073/pnas.1113421108 (2011).
5 Martinez, J. M., R. K.; Lu, Q; Cunha, L. D.; Pelletier, S.; Gingras, S.; Orchard, R.; Guan, J. L; Tan, H.; Peng, J.; Kanneganti, T. D.; Virgin, H. W., and Green, D. R. Molecular characterization of LC3-assMolecular characterization of LC3-associated phagocytosis (LAP) reveals distinct roles for Rubicon, NOX2, and autophagy proteins. Nature cell biology (2015).
6 Henault, J. et al. Noncanonical autophagy is required for type I interferon secretion in response to DNA-immune complexes. Immunity 37, 986-997, doi:10.1016/j.immuni.2012.09.014 (2012).
7 Florey, O., Kim, S. E., Sandoval, C. P., Haynes, C. M. & Overholtzer, M. Autophagy machinery mediates macroendocytic processing and entotic cell death by targeting single membranes. Nature cell biology 13, 1335-1343, doi:10.1038/ncb2363 (2011).
8 Zhou, X. J. et al. Genetic association of PRDM1-ATG5 intergenic region and autophagy with systemic lupus erythematosus in a Chinese population. Annals of the rheumatic diseases 70, 1330-1337, doi:10.1136/ard.2010.140111 (2011).
9 Clarke, A. J. et al. Autophagy is activated in systemic lupus erythematosus and required for plasmablast development. Annals of the rheumatic diseases, doi:10.1136/annrheumdis-2013-204343 (2014).
10 Sanjuan, M. A. et al. Toll-like receptor signalling in macrophages links the autophagy pathway to phagocytosis. Nature 450, 1253-1257 (2007).
11 Gan, B. & Guan, J. L. FIP200, a key signaling node to coordinately regulate various cellular processes. Cell Signal 20, 787-794, doi:10.1016/j.cellsig.2007.10.021 (2008).
12 Mizushima, N. & Levine, B. Autophagy in mammalian development and differentiation. Nature cell biology 12, 823-830, doi:10.1038/ncb0910-823 (2010).
13 Matsunaga, K. et al. Two Beclin 1-binding proteins, Atg14L and Rubicon, reciprocally regulate autophagy at different stages. Nature cell biology 11, 385-396, doi:10.1038/ncb1846 (2009).
14 Clausen, B. E., Burkhardt, C., Reith, W., Renkawitz, R. & Forster, I. Conditional gene targeting in macrophages and granulocytes using LysMcre mice. Transgenic Res 8, 265-277 (1999).
15 Theofilopoulos, A. N. & Dixon, F. J. Murine models of systemic lupus erythematosus. Adv Immunol 37, 269-390 (1985).
16 Ronnblom, L. & Eloranta, M. L. The interferon signature in autoimmune diseases. Current opinion in rheumatology 25, 248-253, doi:10.1097/BOR.0b013e32835c7e32 (2013).
17 Morel, L., Rudofsky, U. H., Longmate, J. A., Schiffenbauer, J. & Wakeland, E. K. Polygenic control of susceptibility to murine systemic lupus erythematosus. Immunity 1, 219-229 (1994).
18 Campbell, A. M., Kashgarian, M. & Shlomchik, M. J. NADPH oxidase inhibits the pathogenesis of systemic lupus erythematosus. Sci Transl Med 4, 157ra141, doi:10.1126/scitranslmed.3004801 (2012).
19 Juncadella, I. J. et al. Apoptotic cell clearance by bronchial epithelial cells critically influences airway inflammation. Nature 493, 547-551, doi:10.1038/nature11714 (2013).
20 Yin, Z. et al. IL-10 regulates murine lupus. Journal of immunology 169, 2148-2155 (2002).
21 Teichmann, L. L. et al. B cell-derived IL-10 does not regulate spontaneous systemic autoimmunity in MRL.Fas(lpr) mice. Journal of immunology 188, 678-685, doi:10.4049/jimmunol.1102456 (2012).
22 Ma, L. et al. Systemic autoimmune disease induced by dendritic cells that have captured necrotic but not apoptotic cells in susceptible mouse strains. Eur J Immunol 35, 3364-3375, doi:10.1002/eji.200535192 (2005).
23 Perry, D., Sang, A., Yin, Y., Zheng, Y. Y. & Morel, L. Murine models of systemic lupus erythematosus. J Biomed Biotechnol 2011, 271694, doi:10.1155/2011/271694 (2011).
24 Houssiau, F. A. et al. Serum interleukin 10 titers in systemic lupus erythematosus reflect disease activity. Lupus 4, 393-395 (1995).
25 Blenman, K. R. et al. IL-10 regulation of lupus in the NZM2410 murine model. Lab Invest 86, 1136-1148, doi:10.1038/labinvest.3700468 (2006).
26 Hampe, J. et al. A genome-wide association scan of nonsynonymous SNPs identifies a susceptibility variant for Crohn disease in ATG16L1. Nature genetics 39, 207-211, doi:10.1038/ng1954 (2007).
27 Lopez, P., Alonso-Perez, E., Rodriguez-Carrio, J. & Suarez, A. Influence of Atg5 mutation in SLE depends on functional IL-10 genotype. PloS one 8, e78756, doi:10.1371/journal.pone.0078756 (2013).
28 Shi, C. S. et al. Activation of autophagy by inflammatory signals limits IL-1beta production by targeting ubiquitinated inflammasomes for destruction. Nature immunology 13, 255-263, doi:10.1038/ni.2215 (2012).
29 De Ravin, S. S. et al. Chronic granulomatous disease as a risk factor for autoimmune disease. The Journal of allergy and clinical immunology 122, 1097-1103, doi:10.1016/j.jaci.2008.07.050 (2008).

### Example 3. Methods for Measuring the Clearance of Cells

### Measuring the Clearance of Dying Cells In Vitro

In order to examine the role of the LAP machinery on the uptake and clearance of dying cells in vitro, bone marrow-derived macrophages were generated from bone marrow progenitors from GFP-LC3+ mice deficient for different components of the LAP pathway, as described. In some cases, macrophages were preloaded with Lysotracker Red, according to the manufacturer's instructions. Macrophages were plated onto fibronectin-coated chamber slides. Apoptosis was induced in wild-type mouse thymocytes by UV irradiation (20 J/m2). After approximately 8 hours, unattached dead cells were labeled with the labeling dye, SytoRed, per manufacturer's instructions, washed twice with PBS and added to macrophage cultures at a ratio of 10:1 (dead cell:macrophage).

Uptake and degradation of dying cells was followed in an environmental control chamber ˜37° C. and 5% CO2. Images were taken at using an oil-immersion Nikon Plan Fluor 40×1.3 N.A. objective with phase contrast optics. GFP-LC3 translocation to and maturation of the dead cell-containing phagosome was quantified by acquiring a time-lapse movie and counting the number of GFP-LC3+ dead cell-containing phagosomes out of the total number of engulfed dead cells for that period. Similarly, the clearance of engulfed dying cells can be determined based on the disappearance of SytoRed (dying cell) fluorescence over time. For each condition, three independent experiments were performed, and the mean with SD error bars was represented.

In order to examine the ability of phagocytes from aged LAP-deficient animals to engulf and translocate GFP-LC3 in vitro, peritoneal exudate cells were elicited from aged GFP-LC3+ mice of different genotypes with 3% Brewer's thioglycollate. After 96 hours, the peritoneum was washed with 10 ml ice cold PBS three times. Cells were collected and washed twice with sterile PBS. Peritoneal exudate cells were resuspended in complete media and allowed to settle for 2 hours (37° C./5% CO2) before co-culture with UV-irradiated wild-type thymocytes (see above). After approximately 8 hours, unattached dead cells were labeled with the labeling dye, CellTrace Violet, per manufacturer's instructions, washed twice with PBS and added to macrophage cultures at a ratio of 10:1 (dead cell:macrophage).

Non-engulfed, non-adherent cells were washed away from the co-culture. The co-cultures were washed once with FACS buffer, and permeabilized with digitonin (Sigma, 200 μg/ml) for 15 minutes on ice. Cells were then washed 3 times with FACS buffer and analyzed by flow cytometry for membrane-bound GFP-LC3-II. This assay removes the soluble, cytosolic form of GFP-LC3 (GFP-LC3-I), while the lipidated, membrane-bound GFP-LC3-II is retained, allowing total GFP fluorescence to be used as a measure of LC3-II generation, indicative of LAP. Permeabilized samples were gated on Singlets/CellTrace Violet+, so as to determine the extent of engulfment and the mean fluorescence intensity (MFI) of GFP-LC3-II associated with cells that had engulfed a CellTrace Violet+ apoptotic thymocyte. Data were acquired using an LSRII cytometer (BD).

### Measuring the Clearance of Dying Cells In Vivo

In order to examine the role of LAP in the response to dying cells in vivo, wild-type thymocytes were labeled with the labeling dye, PKH26, per manufacturer's instructions and washed twice with PBS. Apoptosis was induced in the labeled thymocytes by UV irradiation (20 J/m2), and immediately injected into wild-type animals or animals with LysM-Cre-mediated deficiency of ATG7 (LAP-deficient, autophagy-deficient), LysM-Cre-mediated deficiency of FIP200 (LAP-sufficient, autophagy-deficient), or ubiquitous deletion of Rubicon (LAP-deficient, autophagy-sufficient), all of which also expressed transgenic GFP-LC3.

Spleens, livers, and kidneys were harvested from animals at the indicated time-points, and single cell suspensions were generated. Cells were washed once with FACS buffer, and permeabilized with digitonin (Sigma, 200 μg/ml) for 15 minutes on ice. Cells were then washed 3 times with FACS buffer and analyzed by flow cytometry for membrane-bound GFP-LC3-II. This assay removes the soluble, cytosolic form of GFP-LC3 (GFP-LC3-I), while the lipidated, membrane-bound GFP-LC3-II is retained, allowing total GFP fluorescence to be used as a measure of LC3-II generation, indicative of LAP. Permeabilized samples were first gated on Singlets/PKH26+, so as to determine the mean fluorescence intensity (MFI) of GFP-LC3-II associated with cells that had engulfed a PKH26+ apoptotic thymocyte. To determine which cell types engulfed dying cells, organs were stained with fluorescent antibodies for macrophages (CD11b+ F4/80+), neutrophils (CD11b+ Gr-1+), monocytes (CD11b+ CD115+), and dendritic cells (CD11b+ CD11c+) on ice for 20 minutes. Cells were then washed twice with FACS buffer and analyzed by flow cytometry. Phagocytic efficiency of each cell type (Singlets/cell surface markers+/PKH26+) was quantified by flow cytometry (% PKH26). Data were acquired using an LSRII cytometer (BD).

### Exacerbation of Lupus-Like Syndrome by Continuous Injection of Dying Cells In Vivo

Six-week-old Rubicon+/+ and Rubicon−/− littermates were used. Serum was collected from all animals prior to injection (week 0). 2.0×107 UV-irradiated thymocytes (20 J/m2) suspended in sterile phosphate buffer were injected intravenously into anesthesized mice, once a week for 4 consecutive weeks (from weeks 1 to 4). After a resting period of 15 days, the injections were resumed and carried out for other 2 weeks (weeks 6 and 7). Serum was collected one week after the last injection (week 8) and assessed for levels of anti-dsDNA autoantibodies (Total Ig), anti-nuclear autoantibodies (ANA, Total Ig), and alanine aminotransferase (ALT). At week 8, mice were euthanized; the kidneys were harvested, and stained for immunofluorescence.

### Measuring Phosphatidylserine-Dependent Engulfment In Vitro

L-α-phosphatidylserine (PS) and L-α-phosphatidylcholine (PC) were prepared from either 100% phosphatidylcholine (100% PC) or 70% phosphatidylcholine/30% phosphatidylserine (70% PC/30% PS) and labeled with 25 mg/mL Dextran-Texas Red (Invitrogen). Liposomes were added to bone marrow-derived macrophage GFP-LC3+ cultures (described above) at a ratio of 10:1 (liposomes:macrophage). After incubation, macrophages were washed gently with PBS to remove any non-engulfed liposomes and analyzed for uptake and GFP-LC3 translocation by flow cytometry (described above).

For methods related to measuring phosphatidylserine-dependent engulfment in vivo, please see Fernandez-Boyanapalli, R., et al. (2009) Blood: 113 (9): 2047-2055.

### Example 4. Sample Methods for Measuring LAP Activity

As described in Martinez, J. et al. (Molecular characterization of LC3-associated phagocytosis (LAP) reveals distinct roles for Rubicon, NOX2, and autophagy proteins. Nature cell biology, 17, 893-906.), multiple methods can be used to measure LAP activity.

Cell lysis and immunoblotting. Cells can be lysed in RIPA buffer for 30 min on ice (50 mM Tris, pH 7.5, 150 mM NaCl, 1% Triton X-100, 0.5% DOC, 0.1% SDS, protease inhibitor tablet (Roche), 1 mM NaF, 1 mM Na3VO4 and 1 mM phenylmethylsulphonyl fluoride). After centrifugation (16.1k rcf, 15 min, 4° C.), supernatants can be analysed by SDS-PAGE. Anti-LC3B (catalogue no. ab48394) and anti-UNC93B (catalogue no. ab69497) antibodies can be from abCam. Anti-GATE16 (clone EP4808, catalogue no. TA310512) antibody can be from Origene. Anti-Actin antibody (clone C4, catalogue no. 08691001) can be from MP Biomedicals. Anti-ATG7 (clone D12B11, catalogue no. 8558), anti-Beclin1 (clone D4005, catalogue no. 3495), anti-UVRAG (clone D2Q1Z, catalogue no. 13115), anti-VPS34 (clone D9A5, catalogue no. 4263), anti-Rubicon (clone D9F7, catalogue no. 8465), anti-p-p40PHOX (catalogue no. 4311), anti-ATG14 (catalogue no. 5504), anti-LC3A (clone D50G8, catalogue no. 4599) and anti-GABARAP (clone E1J4E, catalogue no. 13733) antibodies can be from Cell Signaling. p22PHOX (clone C17, catalogue no. 11712) antibody can be from Santa Cruz Biotechnology. Anti-RABS (catalogue no. R4654) and anti-RAB7 (catalogue no. R4779) antibodies can be from Sigma-Aldrich. All primary antibodies, except anti-Actin, were used at a 1:1,000 dilution. Anti-actin antibody was used at 1:10,000. All HRP-conjugated secondary antibodies can be used at a 1:2,000 dilution.

Phagosomes from BMDMs and RAW cells can be obtained as previously described. Briefly, after culture of cells with Pam3csk4-coupled beads, the cells can be washed in cold PBS, pelleted, resuspended in 1 ml of homogenization buffer (250 mM sucrose, 3 mM imidazole, pH 7.4), and homogenized on ice in a Dounce homogenizer. Phagosomes can be isolated by flotation on a sucrose step gradient during centrifugation for 1 h at 100,000 g at 4 C. The latex-bead phagosomal fraction was then collected from the interface of the 10% and 25% sucrose solutions and resuspended in RIPA buffer for protein immunoblot analysis. The entire phagosome purification can be run on 1-2 SDS-PAGE gels owing to the relatively lower protein yield compared with whole-cell lysate samples. Membranes can be sectioned according to the molecular weight marker, and proteins residing within that range of molecular weights were probed with the antibodies listed above. When necessary, membranes can be stripped with Restore PLUS Western Blot Stripping Buffer (Life Technologies), re-blocked in 1×TBST with 5% w/v non-fat dry milk, and probed with fresh antibodies. Images can be captured with an Amersham Imager 600.

Time-lapse imaging and microscopy. Cells can be plated on fibronectin-coated glass-bottom chamber slides (MatTek). Confocal microscopy can be performed using the following systems. Spinning-disc confocal microscopy (SDC) on live cells can be performed with a Marianas SDC imaging system (Intelligent Imaging Innovations/3i) consisting of a CSU22 confocal head (Yokogawa Electric Corporation), DPSS lasers (CrystaLaser) with wavelengths of 445 nm, 473 nm, 523 nm, 561 nm and 658 nm, and a Carl Zeiss 200M motorized inverted microscope (Carl Zeiss Microlmaging), equipped with spherical aberration correction optics (3i). Temperature can be maintained at ˜37 C and 5% CO2 using an environmental control chamber (Solent Scientific). Images can be acquired with a Zeiss Plan-Neofluar 40×1.3 NA DIC objective on a CascadeII 512 EMCCD (Photometrics), using SlideBook 6 software (3i).

Laser scanning confocal microscopy (LSCM) on live cells can be performed with a Nikon TE2000-E inverted microscope equipped with a C1Si confocal system, (Nikon), an argon ion laser at 488 nm and DPSS lasers at 404 nm and 561 nm (Melles Griot). Temperature can be maintained at 37 C and 5% CO2 using an environmental control chamber (InVivo Scientific). Images can be taken at the intervals indicated in the figure legends using an oil-immersion Nikon Plan Fluor 40×1.3 NA objective with phase contrast optics.

Flow cytometry analysis. At the indicated time points, GFP-LC3+ cells can be collected, washed once with FACS buffer, and permeabilized with digitonin (Sigma, 200 g ml-1) for 15 min on ice. Cells can be washed 3 times with FACS buffer and analysed by flow cytometry for membrane-bound GFP-LC3-II. Likewise, PX− mCherry+ cells can be collected, washed once with FACS buffer, and treated with digitonin (200 g ml-1) for 15 min on ice. Cells can then be washed 3 times with FACS buffer and analysed by flow cytometry for membrane-bound PtdIns(3)P.

Quantification of phagocytosis. Phagocytosis can be calculated using flow cytometry analysis (described above). The percentage of phagocytosis equals the number of macrophages that have engulfed Alexa Fluor 594-zymosan or A. fumigatus-dsRed. Quantification of the extent of phagocytosis can be representative of the mean fluorescence intensity (MFI) of the engulfed Alexa Fluor 594-zymosan or A. fumigatus-dsRed.

Class III PI(3)K activity assay. LAPosomes can be purified as known in the art. mVPS34 can be immunoprecipitated and incubated with phosphatidylinositol (PI). The quenched PI(3)K reactions can then be subjected to a Class III PI(3)K Activity Assay (Echelon Biosciences), a competitive ELISA in which the signal is inversely proportional to the amount of PtdIns(3)P produced. Reaction products can be diluted and added to the PtdIns(3)P-coated microplate, for competitive binding to a PtdIns(3)P detector protein. The amount of PtdIns(3)P detector protein bound to the plate can be determined through colorimetric detection. Data (mean±s.d.) represent three independent experiments in which technical triplicates per sample were acquired using a SpectraMax Microplate Reader (Molecular Devices).

Immunofluorescence. Cells grown and stimulated in chamber slides can be fixed with 4% formaldehyde for 20 min at 4 C. Following fixation, cells can be blocked and permeabilized in block buffer (1% BSA, 0.1% Triton X-100 in PBS) for 1 h at room temperature. Cells can be incubated overnight at 4 C with primary antibody diluted 1/200 in block buffer. Cells can be washed extensively in TBS-Tween (Tris-buffered saline containing 0.05% Tween-20) and incubated with Alexa Fluor-conjugated secondary antibodies (Invitrogen). Images can be analysed using an Olympus BX51 FL Microscope and Slidebook software. Alexa Fluor 647-LAMP1 (clone eBio1D4B, catalogue no. 51-1071) antibody was from eBioscience. Anti-oxLDL (catalogue no. bs-1698R) antibody can be from Bioss Antibodies, and anti-PtdIns(3)P (catalogue no. Z-P003) antibody can be from Echelon Biosciences. Anti-LC3B (catalogue no. ab48394) antibody can be from abCam. Anti-Beclin1 (clone D4005, catalogue no. 3495), anti-UVRAG (clone D2Q1Z, catalogue no. 13115), anti-VPS34 (clone D9A5, catalogue no. 4263), anti-Rubicon (clone D9F7, catalogue no. 8465), anti-p-p40PHOX (catalogue no. 4311) and anti-ATG14 (catalogue no. 5504) antibodies can be from Cell Signaling. Anti-ATG7 (catalogue no. A2856) antibody can be from Sigma-Aldrich. p22PHOX (clone C17, catalogue no. 11712) antibody can be from Santa Cruz Biotechnology. All primary antibodies can be used at a 1:100 dilution. All secondary antibodies can be used at 1:400. Representative images from reproducible independent experiments can be shown.

1-34. (canceled)

35. A method of identifying a molecule that modulates LAP activity comprising:
measuring a first level of LAP activity in a cell or tissue;
contacting the cell or tissue with a candidate compound;
measuring a second level of LAP activity of said cell or tissue after said contacting with a candidate compound;
comparing said first level of LAP activity with the second level of LAP activity; and
selecting compounds that modulate the LAP activity.

36. A method of identifying a molecule that modulates LAP activity comprising:
contacting a test cell or tissue with a candidate compound;
measuring a first level of LAP activity of said test cell or tissue after said contacting with a candidate compound;
measuring a second level of LAP activity from a control cell or tissue;
comparing said first level of LAP activity with said second level of LAP activity; and
selecting compounds that modulate the LAP activity.

37. The method of claim 35, wherein compounds are selected that increase or decrease LAP activity.

38. The method of claim 35, wherein measuring said first and second level of LAP activity comprises measuring inflammation.

39. The method of claim 38, wherein measuring inflammation comprises measuring the level of at least one pro-inflammatory or at least one anti-inflammatory cytokine, or a combination of pro-inflammatory and anti-inflammatory cytokines.

40. The method of claim 38, wherein measuring inflammation comprises measuring the level of IL-10, MCP-1, or IL-6.

41. The method of claim 35, wherein said cell or tissue is a bone marrow-derived macrophage or a culture of bone marrow-derived macrophages.

42. The method of claim 41, wherein said bone marrow-derived macrophage is generated from LAP-deficient mice.

43. The method of claim 42, wherein said LAP-deficient mice are Rubicon deficient.

44. The method of claim 35, wherein said selected molecule modulates LAP activity when administered to a subject.

45. The method of claim 44, wherein said subject has an inflammatory disease.

46. A pharmaceutical composition comprising a molecule selected by the method of claim 35.

47. The method of claim 36, wherein compounds are selected that increase or decrease LAP activity.

48. The method of claim 36, wherein measuring said first and second level of LAP activity comprises measuring inflammation.

49. The method of claim 48, wherein measuring inflammation comprises measuring the level of at least one pro-inflammatory or at least one anti-inflammatory cytokine, or a combination of pro-inflammatory and anti-inflammatory cytokines.

50. The method of claim 48, wherein measuring inflammation comprises measuring the level of IL-10, MCP-1, or IL-6.

51. The method of claim 36, wherein said cell or tissue is a bone marrow-derived macrophage or a culture of bone marrow-derived macrophages.

52. The method of claim 51, wherein said bone marrow-derived macrophage is generated from LAP-deficient mice.

53. The method of claim 52, wherein said LAP-deficient mice are Rubicon deficient.

54. A pharmaceutical composition comprising a molecule selected by the method of claim 36.
